(19) United States
(12) Patent Application Publication
(10) Pub. No.: US 2023/0270713 A1
(43) Pub. Date: Aug. 31, 2023

(54) Administration of Resiniferatoxin For Treatment of Bladder Pain or Bladder Cancer

(71) Applicant: Sorrento Therapeutics, Inc., San Diego, CA (US)

(72) Inventors: Alexis Nahama, San Diego, CA (US); Henry Hongjun Ji, Rancho Santa Fe, CA (US)

(73) Assignee: Sorrento Therapeutics, Inc., San Diego, CA (US)

(21) Appl. No.: 18/008,947

(22) PCT Filed: Jun. 18, 2021
(86) PCT No.: PCT/US2021/038038

§ 371 Date: Dec. 7, 2022

Related U.S. Application Data
(60) Provisional application No. 63/120,044, filed on Dec. 1, 2020.
(60) Provisional application No. 63/041,577, filed on Jun. 19, 2020.

(51) Int. Cl. A61K 31/357; A61K 47/26; A61P 35/00; A61P 13/10

(52) U.S. Cl. CPC A61K 31/357; A61K 47/26; A61P 35/00; A61P 13/10

(57) ABSTRACT

Disclosed herein are methods of administering resiniferatoxin (RTX) intravesically for treatment of bladder pain, and compositions for use in such methods.

### I. CROSS REFERENCE TO RELATED APPLICATIONS

This patent application claims priority to U.S. provisional application 63/041,577 filed Jun. 19, 2020, and U.S. provisional application 63/120,044 filed Dec. 1, 2020, the contents of both of which are incorporated herein by reference in their entirety for all purposes.

The present disclosure provides methods of treating bladder pain and/or bladder cancer comprising administering resiniferatoxin (RTX) intravesically, and resiniferatoxin for use in such methods.

### II. INTRODUCTION AND SUMMARY

Bladder pain may occur in a number of conditions, including idiopathic cystitis and bladder cancer. Bladder pain in such conditions can be maladaptive, i.e., pain that does not correlate to a present injury or other external pain source. Misformation of neurons following the onset of the condition resulting in maladaptive pain can result in inappropriate neuronal connections and undesired activity of afferent nociceptive neurons.

Treatment of bladder pain can be challenging. Systemic administration of painkillers is undesirable on a chronic basis due to concerns about side effects and/or dependency/addiction. Meanwhile, administration of pain-suppressing material to the bladder, directly at the site of the nociceptive nerve endings, has shown contradictory results.

Resiniferatoxin (RTX) acts as an ultrapotent analog of capsaicin, the pungent principal ingredient of the red pepper. RTX is a tricyclic diterpene isolated from certain species of Euphorbia. A homovanillyl group is an important structural feature of capsaicin and is the most prominent feature distinguishing resiniferatoxin from typical phorbol-related compounds. Native RTX has the following structure:

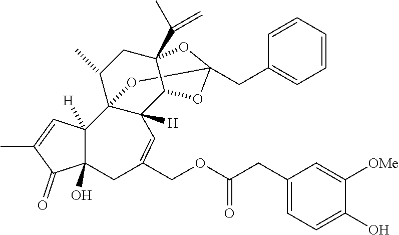

RTX and analog compounds such as tinyatoxin and other compounds (20-homovanillyl esters of diterpenes such as 12-deoxyphorbol 13-phenylacetate 20-homovanillate and mezerein 20-homovanillate) are described in U.S. Pat. Nos. 4,939,194; 5,021,450; and 5,232,684. Other resiniferatoxin-type phorboid vanilloids have also been identified (Szallasi et al. (1999) Brit. J. Pharmacol. 128:428-434).

RTX is known as a TrpV1 agonist. TrpV1, the transient receptor potential cation channel subfamily V member 1 (also known as Vanilloid receptor-1 (VR1)) is a multimeric cation channel prominently expressed in nociceptive primary afferent neurons (Caterina et al. (1997) Nature 389:816-824; Tominaga et al. (1998) Neuron 21:531-543). Activation of TrpV1 typically occurs at the nerve endings via application of painful heat and is up regulated during certain types of inflammatory stimuli. Activation of TrpV1 in peripheral tissues by a chemical agonist results in the opening of calcium channels and the transduction of a pain sensation (Szalllasi et al. (1999) Mol. Pharmacol. 56:581-587). However, direct application of certain TrpV1 agonists to the cell body of a neuron (ganglion) expressing TrpV1 opens calcium channels and triggers a cascade of events leading to programmed cell death (“apoptosis”) (Karai et al. (2004) J. of Clin. Invest. 113:1344-1352).

RTX has been investigated previously for treatment of bladder pain, but the results of such studies were generally contradictory and do not support a clear conclusion as to RTX efficacy for this indication. See, e.g., Mourtzoukou et al. (2008) Int. Urogynecol. J. 19:1571-76 at Abstract (“Six studies provided contradictory results regarding the effectiveness of resiniferatoxin treatment [of interstitial cystitis] . . . . [T]he effectiveness of resiniferatoxin in the treatment of interstitial cystitis remains unknown.”).

For example, in Mourtzoukou et al., supra, a set of studies is summarized at page 1573, Table 1, in which the dosage delivered to the bladder was 3.14 mcg or about 0.05 mcg/kg (i.e., 100 ml of a 50 nM solution, with the molecular weight of RTX being 628.71 g/mol and an average human having a 62 kg mass) or less. Furthermore, discussion of bladder pain in the literature suggests that peripheral changes associated with feline idiopathic cystitis, as a model for human interstitial cystitis/bladder pain syndrome, result from “centrally-mediated sterile inflammation.” See Kullman et al. (2018) Front. Systems Neurosci. 12:13 (15 pages) at 12. Thus, existing teachings indicate that peripheral treatments are likely to be ineffective and that treatment should be directed to the central nervous system (CNS). However, intrathecal and epidural administrations carry a higher degree of risk due, e.g., to proximity to the spinal cord.

Additionally, bladder pain may be maladaptive. Maladaptive pain may arise in any chronic condition in which an inappropriate amount of pain occurs and pain-modulation mechanisms in the central nervous system are implicated, e.g., following chronic or persistent afferent nociceptive neuron activity. Existing publications implicate activity of the dorsal root ganglia and central nervous system in some forms of maladaptive pain including phantom limb pain. See, e.g., Subedi et al., Pain Res. Treatment (2011) 2011:864605, 8 pages (discussing involvement of central neural changes involving cortical reorganization in mechanism of maladaptive phantom limb pain); Borkum, J. Rat-Emo. Cognitive-Behav. Ther. (2010) 28:4-24 (discussing the role of maladaptive cognitions in chronic pain). Current thinking holds that the development of maladaptive pain in general initiates peripherally but results in sensitization in the central nervous system, at which point the sensitization becomes a persistent problem against which peripheral therapies are not expected to be successful. Taken together, these publications lead to the expectation that treatments focused on elements of the nervous system peripheral to the dorsal root ganglia, without treating the dorsal root ganglia or central nervous system, may have low or less efficacy than treatments targeting the dorsal root ganglia or central nervous system. These teachings dovetail with the findings regarding RTX delivery to the bladder discussed above to show that existing methods are inadequate.

Accordingly, there is a need for improved methods and uses of RTX for treatment of bladder pain. The present disclosure aims to meet this need and/or provide other benefits.

Notably, it is recognized herein that increased dosages relative to those used in previous studies more effectively ablate neurons and therefore are considered more effective. Provided herein, for example, are methods of administering RTX intravesically for treatment of bladder pain to a subject in need thereof, wherein the RTX is administered at a dose of at least about 10 mcg or at least about 0.1 mcg/kg, which can provide greater efficacy than existing methods.

There is also a need for improved and/or alternative treatment options for bladder cancer. As described herein, it has been found that RTX treatment can provide beneficial effects against bladder cancer.

Accordingly, the following exemplary embodiments are provided. Embodiment 1 is a method of treating bladder pain or bladder cancer, comprising intravesically administering resiniferatoxin (RTX) to a subject in need of treatment of bladder pain or bladder cancer, wherein the RTX is administered at a dose of at least about 10 mcg or at least about 0.1 mcg/kg, or the RTX is administered at a concentration and volume such that the intravesical concentration of RTX in the bladder is at least about 0.1 mcg/ml.

Embodiment 2 is a composition comprising resiniferatoxin (RTX) for use in a method of treating bladder pain or bladder cancer, the method comprising intravesically administering RTX to a subject in need of treatment of bladder pain or bladder cancer at a dose of at least about 10 mcg or at least about 0.1 mcg/kg, or the RTX is administered at a concentration and volume such that the intravesical concentration of RTX in the bladder is at least about 0.1 mcg/ml.

Embodiment 3 is the method or composition for use according to embodiment 1 or 2, wherein the bladder pain comprises maladaptive bladder pain.

Embodiment 4 is the method or composition for use of any one of the preceding embodiments, wherein the treatment reduces local and central effects of the maladaptive pain.

Embodiment 5 is the method or composition for use according to any one of the preceding embodiments, wherein the subject has cystitis.

Embodiment 6 is the method or composition for use according to any one of the preceding embodiments, wherein the subject has idiopathic cystitis.

Embodiment 7 is the method or composition for use according to any one of the preceding embodiments, wherein the subject has bladder cancer.

Embodiment 8 is the method or composition for use according to any one of the preceding embodiments, wherein the bladder pain comprises neuropathic bladder pain.

Embodiment 9 is the method or composition for use according to any one of the preceding embodiments, wherein the bladder pain results from or is associated with stress-based activation of C fibers.

Embodiment 10 is the method or composition for use according to any one of the preceding embodiments, wherein the subject previously underwent bladder surgery.

Embodiment 11 is the method or composition for use according to any one of the preceding embodiments, wherein the bladder pain is subsequent to an injury, which is optionally an injury to the spine or lower back, such as a spinal disk injury.

Embodiment 12 is the method or composition for use according to any one of the preceding embodiments, wherein the subject has experienced a bacterial bladder infection, or a plurality of bacterial bladder infections.

Embodiment 13 is the method or composition for use according to any one of the preceding embodiments, wherein the subject has a hyperreactive bladder and/or a lowered threshold for bladder contraction and/or urge to urinate.

Embodiment 14 is the method or composition for use according to any one of the preceding embodiments, wherein the method comprises administering RTX at a concentration of 0.1 mcg/ml-0.2 mcg/ml, 0.2 mcg/ml-0.3 mcg/ml, 0.3 mcg/ml-0.4 mcg/ml, 0.4 mcg/ml-0.5 mcg/ml, 0.5 mcg/ml-0.6 mcg/ml, 0.6 mcg/ml-0.7 mcg/ml, 0.7 mcg/ml-0.8 mcg/ml, 0.8 mcg/ml-0.9 mcg/ml, 0.9 mcg/ml-1.0 mcg/ml, 1.0 mcg/ml-1.1 mcg/ml, 1.1 mcg/ml-1.2 mcg/ml, 1.2 mcg/ml-1.3 mcg/ml, 1.3 mcg/ml-1.4 mcg/ml, 1.4 mcg/ml-1.5 mcg/ml, 1.5 mcg/ml-2 mcg/ml, 2 mcg/ml-3 mcg/ml, 3 mcg/ml-4 mcg/ml, 4 mcg/ml-5 mcg/ml, 5 mcg/ml-6 mcg/ml, 6 mcg/ml-7 mcg/ml, 7 mcg/ml-8 mcg/ml, 8 mcg/ml-9 mcg/ml, 9 mcg/ml-10 mcg/ml, 10 mcg/ml-11 mcg/ml, 11 mcg/ml-12 mcg/ml, 12 mcg/ml-13 mcg/ml, 13 mcg/ml-14 mcg/ml, or 14 mcg/ml-15 mcg/ml.

Embodiment 14a is the method or composition for use of embodiment 14, wherein the method comprises administering RTX at a concentration of 0.3 mcg/ml-0.4 mcg/ml. Embodiment 14b is the method or composition for use of embodiment 14, wherein the method comprises administering RTX at a concentration of 0.4 mcg/ml-0.5 mcg/ml. Embodiment 14c is the method or composition for use of embodiment 14, wherein the method comprises administering RTX at a concentration of 0.5 mcg/ml-0.6 mcg/ml. Embodiment 14d is the method or composition for use of embodiment 14, wherein the method comprises administering RTX at a concentration of 0.6 mcg/ml-0.7 mcg/ml. Embodiment 14e is the method or composition for use of embodiment 14, wherein the method comprises administering RTX at a concentration of 0.7 mcg/ml-0.8 mcg/ml. Embodiment 14f is the method or composition for use of embodiment 14, wherein the method comprises administering RTX at a concentration of 0.8 mcg/ml-0.9 mcg/ml. Embodiment 14g is the method or composition for use of embodiment 14, wherein the method comprises administering RTX at a concentration of 0.9 mcg/ml-1.0 mcg/ml. Embodiment 14h is the method or composition for use of embodiment 14, wherein the method comprises administering RTX at a concentration of 1.0 mcg/ml-1.1 mcg/ml. Embodiment 14i is the method or composition for use of embodiment 14, wherein the method comprises administering RTX at a concentration of 1.1 mcg/ml-1.2 mcg/ml. Embodiment 14j is the method or composition for use of embodiment 14, wherein the method comprises administering RTX at a concentration of 1.2 mcg/ml-1.3 mcg/ml. Embodiment 14k is the method or composition for use of embodiment 14, wherein the method comprises administering RTX at a concentration of 1.3 mcg/ml-1.4 mcg/ml. Embodiment 14l is the method or composition for use of embodiment 14, wherein the method comprises administering RTX at a concentration of 1.4 mcg/ml-1.5 mcg/ml. Embodiment 14m is the method or composition for use of embodiment 14, wherein the method comprises administering RTX at a concentration of 1.5 mcg/ml-2 mcg/ml. Embodiment 14n is the method or composition for use of embodiment 14, wherein the method comprises administering RTX at a concentration of 2 mcg/ml-3 mcg/ml. Embodiment 14o is the method or composition for use of embodiment 14, wherein the method comprises administering RTX at a concentration of 3 mcg/ml-4 mcg/ml. Embodiment 14p is the method or composition for use of embodiment 14, wherein the method comprises administering RTX at a concentration of 4 mcg/ml-5 mcg/ml. Embodiment 14q is the method or composition for use of embodiment 14, wherein the method comprises administering RTX at a concentration of 5 mcg/ml-6 mcg/ml. Embodiment 14r is the method or composition for use of embodiment 14, wherein the method comprises administering RTX at a concentration of 6 mcg/ml-7 mcg/ml. Embodiment 14s is the method or composition for use of embodiment 14, wherein the method comprises administering RTX at a concentration of 7 mcg/ml-8 mcg/ml. Embodiment 14t is the method or composition for use of embodiment 14, wherein the method comprises administering RTX at a concentration of 8 mcg/ml-9 mcg/ml. Embodiment 14u is the method or composition for use of embodiment 14, wherein the method comprises administering RTX at a concentration of 9 mcg/ml-10 mcg/ml. Embodiment 14v is the method or composition for use of embodiment 14, wherein the method comprises administering RTX at a concentration of 10 mcg/ml-11 mcg/ml. Embodiment 14w is the method or composition for use of embodiment 14, wherein the method comprises administering RTX at a concentration of 11 mcg/ml-12 mcg/ml. Embodiment 14x is the method or composition for use of embodiment 14, wherein the method comprises administering RTX at a concentration of 12 mcg/ml-13 mcg/ml. Embodiment 14y is the method or composition for use of embodiment 14, wherein the method comprises administering RTX at a concentration of 13 mcg/ml-14 mcg/ml. Embodiment 14z is the method or composition for use of embodiment 14, wherein the method comprises administering RTX at a concentration of 14 mcg/ml-15 mcg/ml.

Embodiment 15 is the method or composition for use according to any one of the preceding embodiments, wherein a dose of 10 mcg-20 mcg, 20 mcg-30 mcg, 30 mcg-40 mcg, 40 mcg-50 mcg, 50 mcg-60 mcg, 60 mcg-70 mcg, 70 mcg-80 mcg, 80 mcg-90 mcg, 90 mcg-100 mcg, 100 mcg-110 mcg, 110 mcg-120 mcg, 120 mcg-130 mcg, 130 mcg-140 mcg, 140 mcg-150 mcg, 150 mcg-160 mcg, 160 mcg-170 mcg, 170 mcg-180 mcg, 180 mcg-190 mcg, 190 mcg-200 mcg, 200 mcg-210 mcg, 210 mcg-220 mcg, 220 mcg-230 mcg, 230 mcg-240 mcg, 240 mcg-250 mcg, 250 mcg-260 mcg, 260 mcg-270 mcg, 270 mcg-280 mcg, 280 mcg-290 mcg, or 290 mcg-300 mcg of RTX is administered intravesically.

Embodiment 15a is the method or composition for use of embodiment 15, wherein the dose of RTX administered intravesically is 10 mcg-20 mcg. Embodiment 15b is the method or composition for use of embodiment 15, wherein the dose of RTX administered intravesically is 20 mcg-30 mcg. Embodiment 15c is the method or composition for use of embodiment 15, wherein the dose of RTX administered intravesically is 30 mcg-40 mcg. Embodiment 15d is the method or composition for use of embodiment 15, wherein the dose of RTX administered intravesically is 40 mcg-50 mcg. Embodiment 15e is the method or composition for use of embodiment 15, wherein the dose of RTX administered intravesically is 50 mcg-60 mcg. Embodiment 15f is the method or composition for use of embodiment 15, wherein the dose of RTX administered intravesically is 60 mcg-70 mcg. Embodiment 15g is the method or composition for use of embodiment 15, wherein the dose of RTX administered intravesically is 70 mcg-80 mcg. Embodiment 15h is the method or composition for use of embodiment 15, wherein the dose of RTX administered intravesically is 80 mcg-90 mcg. Embodiment 15i is the method or composition for use of embodiment 15, wherein the dose of RTX administered intravesically is 90 mcg-100 mcg. Embodiment 15j is the method or composition for use of embodiment 15, wherein the dose of RTX administered intravesically is 100 mcg-110 mcg. Embodiment 15k is the method or composition for use of embodiment 15, wherein the dose of RTX administered intravesically is 110 mcg-120 mcg. Embodiment 15l is the method or composition for use of embodiment 15, wherein the dose of RTX administered intravesically is 120 mcg-130 mcg. Embodiment 15m is the method or composition for use of embodiment 15, wherein the dose of RTX administered intravesically is 130 mcg-140 mcg. Embodiment 15n is the method or composition for use of embodiment 15, wherein the dose of RTX administered intravesically is 140 mcg-150 mcg. Embodiment 15o is the method or composition for use of embodiment 15, wherein the dose of RTX administered intravesically is 150 mcg-160 mcg. Embodiment 15p is the method or composition for use of embodiment 15, wherein the dose of RTX administered intravesically is 160 mcg-170 mcg. Embodiment 15q is the method or composition for use of embodiment 15, wherein the dose of RTX administered intravesically is 170 mcg-180 mcg. Embodiment 15r is the method or composition for use of embodiment 15, wherein the dose of RTX administered intravesically is 180 mcg-190 mcg. Embodiment 15s is the method or composition for use of embodiment 15, wherein the dose of RTX administered intravesically is 190 mcg-200 mcg. Embodiment 15t is the method or composition for use of embodiment 15, wherein the dose of RTX administered intravesically is 200 mcg-210 mcg. Embodiment 15u is the method or composition for use of embodiment 15, wherein the dose of RTX administered intravesically is 210 mcg-220 mcg. Embodiment 15v is the method or composition for use of embodiment 15, wherein the dose of RTX administered intravesically is 220 mcg-230 mcg. Embodiment 15x is the method or composition for use of embodiment 15, wherein the dose of RTX administered intravesically is 230 mcg-240 mcg. Embodiment 15y is the method or composition for use of embodiment 15, wherein the dose of RTX administered intravesically is 240 mcg-250 mcg. Embodiment 15z is the method or composition for use of embodiment 15, wherein the dose of RTX administered intravesically is 250 mcg-260 mcg. Embodiment 15aa is the method or composition for use of embodiment 15, wherein the dose of RTX administered intravesically is 260 mcg-270 mcg. Embodiment 15bb is the method or composition for use of embodiment 15, wherein the dose of RTX administered intravesically is 270 mcg-280 mcg. Embodiment 15cc is the method or composition for use of embodiment 15, wherein the dose of RTX administered intravesically is 280 mcg-290 mcg. Embodiment 15dd is the method or composition for use of embodiment 15, wherein the dose of RTX administered intravesically is 290 mcg-300 mcg.

Embodiment 16 is the method or composition for use according to any one of the preceding embodiments, wherein a dose of 0.1 mcg/kg-0.2 mcg/kg, 0.2 mcg/kg-0.3 mcg/kg, 0.3 mcg/kg-0.4 mcg/kg, 0.4 mcg/kg-0.5 mcg/kg, 0.5 mcg/kg-0.6 mcg/kg, 0.6 mcg/kg-0.7 mcg/kg, 0.7 mcg/kg-0.8 mcg/kg, 0.8 mcg/kg-0.9 mcg/kg, 0.9 mcg/kg-1 mcg/kg, 1 mcg/kg-1.2 mcg/kg, 1.2 mcg/kg-1.4 mcg/kg, 1.4 mcg/kg-1.6 mcg/kg, 1.6 mcg/kg-1.8 mcg/kg, 1.8 mcg/kg-2.0 mcg/kg, 2.0 mcg/kg-2.2 mcg/kg, 2.2 mcg/kg-2.4 mcg/kg, 2.4 mcg/kg-2.6 mcg/kg, 2.6 mcg/kg-2.8 mcg/kg, 2.8 mcg/kg-3.0 mcg/kg, 3.0 mcg/kg-3.2 mcg/kg, 3.2 mcg/kg-3.4 mcg/kg, 3.4 mcg/kg-3.6 mcg/kg, 3.6 mcg/kg-3.8 mcg/kg, 4.0 mcg/kg-4.2 mcg/kg, 4.2 mcg/kg-4.4 mcg/kg, 4.4 mcg/kg-4.6 mcg/kg, 4.6 mcg/kg-4.8 mcg/kg, 4.8 mcg/kg-5.0 mcg/kg, 5.0 mcg/kg-5.2 mcg/kg, 5.2 mcg/kg-5.4 mcg/kg, 5.4 mcg/kg-5.6 mcg/kg, 5.6 mcg/kg-5.8 mcg/kg, or 5.8 mcg/kg-6.0 mcg/kg of RTX is administered intravesically.

Embodiment 16a is the method or composition for use of embodiment 16, wherein the dose of RTX administered intravesically is 0.1 mcg/kg-0.2 mcg/kg. Embodiment 16b is the method or composition for use of embodiment 16, wherein the dose of RTX administered intravesically is 0.2 mcg/kg-0.3 mcg/kg. Embodiment 16c is the method or composition for use of embodiment 16, wherein the dose of RTX administered intravesically is 0.3 mcg/kg-0.4 mcg/kg. Embodiment 16d is the method or composition for use of embodiment 16, wherein the dose of RTX administered intravesically is 0.4 mcg/kg-0.5 mcg/kg. Embodiment 16e is the method or composition for use of embodiment 16, wherein the dose of RTX administered intravesically is 0.5 mcg/kg-0.6 mcg/kg. Embodiment 16f is the method or composition for use of embodiment 16, wherein the dose of RTX administered intravesically is 0.6 mcg/kg-0.7 mcg/kg. Embodiment 16g is the method or composition for use of embodiment 16, wherein the dose of RTX administered intravesically is 0.7 mcg/kg-0.8 mcg/kg. Embodiment 16h is the method or composition for use of embodiment 16, wherein the dose of RTX administered intravesically is 0.8 mcg/kg-0.9 mcg/kg. Embodiment 16i is the method or composition for use of embodiment 16, wherein the dose of RTX administered intravesically is 0.9 mcg/kg-1 mcg/kg. Embodiment 16j is the method or composition for use of embodiment 16, wherein the dose of RTX administered intravesically is 1 mcg/kg-1.2 mcg/kg. Embodiment 16k is the method or composition for use of embodiment 16, wherein the dose of RTX administered intravesically is 1.2 mcg/kg-1.4 mcg/kg. Embodiment 16l is the method or composition for use of embodiment 16, wherein the dose of RTX administered intravesically is 1.4 mcg/kg-1.6 mcg/kg. Embodiment 16m is the method or composition for use of embodiment 16, wherein the dose of RTX administered intravesically is 1.6 mcg/kg-1.8 mcg/kg. Embodiment 16n is the method or composition for use of embodiment 16, wherein the dose of RTX administered intravesically is 1.8 mcg/kg-2.0 mcg/kg.

Embodiment 17 is the method or composition for use according to any one of the preceding embodiments, wherein the RTX is delivered in a composition having a volume of 1 ml-10 ml, 10 ml-20 ml, 20 ml-30 ml, 30 ml-40 ml, 40 ml-50 ml, 50 ml-60 ml, 60 ml-70 ml, 70 ml-80 ml, 80 ml-90 ml, 90 ml-100 ml, 100 ml-110 ml, 110 ml-120 ml, 120 ml-130 ml, 130 ml-140 ml, 140 ml-150 ml, 160 ml-170 ml, 170 ml-180 ml, 180 ml-190 ml, 190 ml-200 ml, 200 ml-210 ml, 210 ml-220 ml, 220 ml-230 ml, 230 ml-240 ml, 240 ml-250 ml, 260 ml-270 ml, 270 ml-280 ml, 280 ml-290 ml, or 290 ml-300 ml.

Embodiment 17a is the method or composition for use of embodiment 17, wherein the RTX is delivered in a composition having a volume of 20 ml-30 ml. Embodiment 17b is the method or composition for use of embodiment 17, wherein the RTX is delivered in a composition having a volume of 30 ml-40 ml. Embodiment 17c is the method or composition for use of embodiment 17, wherein the RTX is delivered in a composition having a volume of 40 ml-50 ml. Embodiment 17d is the method or composition for use of embodiment 17, wherein the RTX is delivered in a composition having a volume of 50 ml-60 ml. Embodiment 17e is the method or composition for use of embodiment 17, wherein the RTX is delivered in a composition having a volume of 60 ml-70 ml. Embodiment 17f is the method or composition for use of embodiment 17, wherein the RTX is delivered in a composition having a volume of 70 ml-80 ml. Embodiment 17g is the method or composition for use of embodiment 17, wherein the RTX is delivered in a composition having a volume of 80 ml-90 ml. Embodiment 17h is the method or composition for use of embodiment 17, wherein the RTX is delivered in a composition having a volume of 90 ml-100 ml. Embodiment 17i is the method or composition for use of embodiment 17, wherein the RTX is delivered in a composition having a volume of 100 ml-110 ml. Embodiment 17j is the method or composition for use of embodiment 17, wherein the RTX is delivered in a composition having a volume of 110 ml-120 ml. Embodiment 17k is the method or composition for use of embodiment 17, wherein the RTX is delivered in a composition having a volume of 120 ml-130 ml. Embodiment 17m is the method or composition for use of embodiment 17, wherein the RTX is delivered in a composition having a volume of 130 ml-140 ml. Embodiment 17n is the method or composition for use of embodiment 17, wherein the RTX is delivered in a composition having a volume of or 140 ml-150 ml. Embodiment 17o is the method or composition for use of embodiment 17, wherein the RTX is delivered in a composition having a volume of 1 ml-10 ml. Embodiment 17p is the method or composition for use of embodiment 17, wherein the RTX is delivered in a composition having a volume of 10 ml-20 ml.

Embodiment 18 is the method or composition for use according to any one of the preceding embodiments, wherein the RTX is administered at a concentration and volume such that the intravesical concentration of RTX in the bladder is at least about 0.1 mcg/ml, such as 0.1 mcg/ml-0.2 mcg/ml, 0.2 mcg/ml-0.3 mcg/ml, 0.3 mcg/ml-0.4 mcg/ml, 0.4 mcg/ml-0.5 mcg/ml, 0.5 mcg/ml-0.6 mcg/ml, 0.6 mcg/ml-0.7 mcg/ml, 0.7 mcg/ml-0.8 mcg/ml, 0.8 mcg/ml-0.9 mcg/ml, or 0.9 mcg/ml-1 mcg/ml.

Embodiment 19 is the method or composition for use according to any one of the preceding embodiments, wherein the RTX is administered at a concentration and volume such that the intravesical concentration of RTX in the bladder is at least about 1 mcg/ml, e.g., 1 mcg/ml-1.1 mcg/ml, 1.1 mcg/ml-1.2 mcg/ml, 1.2 mcg/ml-1.3 mcg/ml, 1.3 mcg/ml-1.4 mcg/ml, 1.4 mcg/ml-1.5 mcg/ml, 1.5 mcg/ml-1.6 mcg/ml, 1.6 mcg/ml-1.7 mcg/ml, 1.7 mcg/ml-1.8 mcg/ml, 1.8 mcg/ml-1.9 mcg/ml, or 1.9 mcg/ml-2 mcg/ml.

Embodiment 20 is the method or composition for use of any one of the preceding embodiments, wherein the RTX is administered at a concentration of at least 0.25 mcg/ml.

Embodiment 21 is the method or composition for use of any one of the preceding embodiments, wherein the RTX is administered at a concentration of at least 0.3 mcg/ml.

Embodiment 22 is the method or composition for use of any one of the preceding embodiments, wherein the RTX is administered at a concentration of at least 0.4 mcg/ml.

Embodiment 23 is the method or composition for use of any one of the preceding embodiments, wherein the RTX is administered at a concentration of at least 0.5 mcg/ml.

Embodiment 23a is the method or composition for use of any one of the preceding embodiments, wherein the RTX is administered at a concentration of at least 1.0 mcg/ml.

Embodiment 23b is the method or composition for use of any one of the preceding embodiments, wherein the RTX is administered at a concentration of at least 2.0 mcg/ml.

Embodiment 24 is the method or composition for use of any one of the preceding embodiments, wherein the subject is a mammal.

Embodiment 25 is the method or composition for use of embodiment 24, wherein the mammal is a cat or dog.

Embodiment 26 is the method or composition for use of embodiment 24, wherein the mammal is a human.

Embodiment 27 is the method or composition for use of embodiment 26, wherein the RTX is delivered in a composition having a volume of 50 ml-60 ml, 60 ml-70 ml, 70 ml-80 ml, 80 ml-90 ml, 90 ml-100 ml, 100 ml-110 ml, 110 ml-120 ml, 120 ml-130 ml, 130 ml-140 ml, 140 ml-150 ml, 160 ml-170 ml, 170 ml-180 ml, 180 ml-190 ml, 190 ml-200 ml, 200 ml-210 ml, 210 ml-220 ml, 220 ml-230 ml, 230 ml-240 ml, or 240 ml-250 ml.

Embodiment 28 is the method or composition for use according to any one of the preceding embodiments, wherein the method comprises administering a pharmaceutical formulation comprising the RTX and a pharmaceutically acceptable carrier.

Embodiment 29 is the method or composition for use of embodiment 28, wherein the pharmaceutically acceptable carrier comprises water.

Embodiment 30 is the method or composition for use of embodiment 27 or 28, wherein the pharmaceutically acceptable carrier comprises polysorbate 80.

Embodiment 31 is the method or composition for use of any one of embodiments 28-30, wherein the pharmaceutically acceptable carrier comprises a buffer, optionally wherein the buffer is phosphate buffer and/or the pH of the formulation is about 7.0-7.5 or about 7.2.

### III. BRIEF DESCRIPTION OF THE DRAWINGS

FIG. 1A-1H show confocal microscopy images of calcitonin gene related peptide (CGRP) positive axons (panels A-D) and Substance P (SP) positive axons (panels E-H) in cat bladders at day 14 post RTX treatment with single doses of 5, 25, and 50 micrograms (mcg). Intravesical RTX treatment decreased the density of both CGRP and SP-immunoreactive axons innervating cat bladder body at 25 and 50 mcg doses when compared to control saline treatment.

FIG. 2A-2F show the density of afferent nerve fibers (mm/mm³) in individual cat bladders as revealed by immunohistochemistry (IHC). The density of CGRP nerve fibers is shown in the body (FIG. 2A), trigon (FIG. 2B), and fundus (FIG. 2C) of the bladder. The density of SP nerve fibers is also shown in the body (FIG. 2D), trigon (FIG. 2E), and fundus (FIG. 2F). Each bar represents a single cat that received the control saline treatment or the specified dose of RTX.

FIG. 3A-3F show the density of afferent nerve fibers (mm/mm³) in cat bladders from each RTX treatment group. The density of CGRP nerve fibers is shown in the body (FIG. 3A), trigon (FIG. 3B), and fundus (FIG. 3C) of the bladder. The density of SP nerve fibers is shown in the body (FIG. 3D), trigon (FIG. 3E), and fundus (FIG. 3D). Each bar represents the mean density of fibers±S.E.M.

FIG. 4A-4D show representative IHC confocal microscopy images of neurons from sacral dorsal root ganglia (S2 DRG) of a control cat treated with saline or a cat treated with a single 25 mcg dose of RTX. Expression of CGRP and SP in the control animal are shown in FIGS. 4A and 4C respectively, and in FIGS. 4B and 4D for the RTX treated cat. RTX treatment did not modify the number of neurons expressing these markers when compared to control.

FIG. 5A-5D show the percentage of CGRP and SP positive neurons in the S1 and S2 dorsal root ganglion (DRG) of control and RTX treated cats, 14 days post treatment. RTX at doses of 5, 25 and 50 mcg did not affect the percentage of CGRP+ neurons in the S1 DRG (FIG. 5A) or in the S2 DRG (FIG. 5B) when compared to control. The percentage of SP+ neurons in both the S1 DRG (FIG. 5C) and the S2 DRG (FIG. 5D) was comparable to the control at 14 days post-treatment. Each bar represents the mean±SEM.

FIG. 6 shows representative confocal microscopy imaging of CGRP+ axons innervating a healthy dog bladder.

FIG. 7 shows representative confocal microscopy imaging of SP+ axons innervating a healthy dog bladder.

FIG. 8A-8F show the density of CGRP+ (FIG. 8A-8C) and SP+ (FIG. 8D-8F) axons innervating dog bladder from control (FIGS. 8A and 8D), dog bladder treated with a single intravesical application of RTX at a dose of 25 mcg (FIGS. 8B and 8E) or 250 mcg (FIGS. 8C and 8F).

FIG. 9A-9C show the density of afferent nerve fibers (mm/mm³) in control and RTX treated dog bladders as revealed by immunohistochemistry (IHC). The density of CGRP+ nerve fibers is shown in the body (FIG. 9A), fundus (FIG. 9B), and trigon (FIG. 9C) of the bladder. Each bar represents the mean±SEM when n≥2.

FIG. 10A-10C show the density of afferent nerve fibers (mm/mm³) in control and RTX treated dog bladders as revealed by immunohistochemistry (IHC). The density of SP+ nerve fibers is shown in the body (FIG. 10A), fundus (FIG. 10B), and trigon (FIG. 10C), and fundus. Each bar represents the mean±SEM when n≥2.

FIG. 11A-FIG. 11I show representative sections of dog bladder fundus stained with H&E and evaluated for histopathology. Control dog bladder is shown FIGS. 11A, 11D, and 10G. Dog bladder treated with 25 mcg RTX is shown in FIGS. 11B, 11E, and 10H. Dog bladder treated with 250 mcg RTX is shown in FIGS. 11C, 11F, and 11I.

### IV. DETAILED DESCRIPTION OF CERTAIN EMBODIMENTS

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### A. Definitions

“Intravesically” administering an agent refers to delivering the agent to the interior of the urinary bladder so that it contacts the interior surface of the bladder. Intravesical administration may be accomplished, e.g., using a catheter. Alternatively, an agent can be delivered intravesically by injection through the abdominal wall and into the bladder.

“Bladder cancer” refers to any condition in which malignant cells are present in the urinary bladder.

“Pain associated with bladder cancer” refers to painful sensations from the bladder in a subject with bladder cancer, wherein the sensations do not result from a cause or set of causes wholly unrelated to the cancer. Painful sensations from the bladder that result in part from bladder cancer and in part from an unrelated cause are considered associated with bladder cancer.

“Maladaptive pain” refers to pain disproportionate to actual tissue damage that persists after the tissue has healed and/or in the absence of proportionate tissue damage so that the pain itself is a problem apart from any underlying current source of pain such as an injury. Maladaptive pain is distinct from neuropathic pain.

“Neuropathic pain” refers to pain that results from damage or disease affecting sensory neurons.

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

### B. Exemplary Methods and Compositions for Use

Provided herein are methods for treating bladder pain, comprising intravesically administering resiniferatoxin (RTX) to a subject in need of treatment of bladder pain. Also provided are compositions comprising RTX for use in a method of treating bladder pain, the method comprising intravesically administering RTX to a subject in need of treatment of bladder pain, wherein the RTX is administered at a dose of at least about 10 mcg or at least about 0.1 mcg/kg. The present disclosure is based in part on the realization that, despite statements in the literature (e.g., in Mourtzoukou et al., supra) questioning the effectiveness of RTX for treatment of bladder pain, administering RTX as described herein can effectively ablate nociceptive nerve endings or nerve fibers in the bladder, which is considered indicative of effective pain treatment.

Bladder pain can arise in a number of different ways. In some embodiments, the bladder pain treated herein is maladaptive. The maladaptive pain may arise subsequent to surgery on the bladder. In some embodiments, the bladder pain treated herein is neuropathic. Bladder pain may result from or be associated with stress-based activation of C fibers. Bladder pain may result from or be associated with idiopathic cystitis (e.g., sterile idiopathic cystitis). Bladder pain may occur subsequent to an injury, e.g., an injury to the spine or lower back (e.g., a spinal disk injury) that sensitizes the bladder and/or renders it more reactive. Recurrent infections from low level bacterial colonization of the bladder and/or associated immune activity may contribute to bladder pain or render the nerve endings in the bladder more sensitive to pain. In some embodiments, the bladder pain is in an individual with a hyperreactive bladder (which may be related to a spinal injury), and/or a lowered threshold for bladder contraction and/or urge to urinate. The treatments of bladder pain described herein can be applied to treat any of the foregoing forms of bladder pain.

With respect to maladaptive pain, although the literature suggests that the central nervous system plays a substantial role in maladaptive pain including the origin thereof, administration of RTX intravesically to treat bladder pain can provide significant relief. Without wishing to be bound by any particular theory, intravesically administering RTX may interrupt signals carried by afferent nociceptive neurons to a sufficient degree and for a sufficient duration to provide not only local but also central neurological effects that result in long-term reduction or control of maladaptive pain without the need for direct treatment of the dorsal root ganglia or central nervous system, contrary to the notion that maladaptive pain involves sensitization in the central nervous system as a persistent problem unlikely to be addressed through peripheral treatments. Thus, administration of RTX to treat maladaptive pain intravesically as disclosed herein may provide benefits that could not have been predicted from the literature, such as allowing effective pain relief without treatment of the dorsal root ganglia or central nervous system or systemic treatment and the attendant risks thereof, and/or with reduced frequency relative to other treatments.

The compositions and methods described herein are for use with any subject in whom RTX is effective, e.g., able to bind and activate TrpV1 or a homolog thereof, and who is in need of treatment for bladder pain. In some embodiments, the subject is a mammal. In some embodiments, the mammal is a human. In some embodiments, the mammal is a cat. In some embodiments, the mammal is a dog.

In some embodiments, the subject has bladder cancer. In some embodiments, the subject has cystitis, such as idiopathic cystitis or interstitial cystitis.

RTX may be administered intravesically using a catheter, such as a Foley catheter.

### 1. Dosage

In some embodiments, the RTX is administered at a dose of at least 10 mcg, such as 10 mcg-20 mcg, 20 mcg-30 mcg, 30 mcg-40 mcg, 40 mcg-50 mcg, 50 mcg-60 mcg, 60 mcg-70 mcg, 70 mcg-80 mcg, 80 mcg-90 mcg, 90 mcg-100 mcg, 100 mcg-110 mcg, 110 mcg-120 mcg, 120 mcg-130 mcg, 130 mcg-140 mcg, 140 mcg-150 mcg, 150 mcg-160 mcg, 160 mcg-170 mcg, 170 mcg-180 mcg, 180 mcg-190 mcg, 190 mcg-200 mcg, 200 mcg-210 mcg, 210 mcg-220 mcg, 220 mcg-230 mcg, 230 mcg-240 mcg, 240 mcg-250 mcg, 250 mcg-260 mcg, 260 mcg-270 mcg, 270 mcg-280 mcg, 280 mcg-290 mcg, or 290 mcg-300 mcg.

In some embodiments, the RTX is administered at a dose of at least about 0.1 mcg/kg, such as 0.1 mcg/kg-0.2 mcg/kg, 0.2 mcg/kg-0.3 mcg/kg, 0.3 mcg/kg-0.4 mcg/kg, 0.4 mcg/kg-0.5 mcg/kg, 0.5 mcg/kg-0.6 mcg/kg, 0.6 mcg/kg-0.7 mcg/kg, 0.7 mcg/kg-0.8 mcg/kg, 0.8 mcg/kg-0.9 mcg/kg, 0.9 mcg/kg-1 mcg/kg, 1 mcg/kg-1.2 mcg/kg, 1.2 mcg/kg-1.4 mcg/kg, 1.4 mcg/kg-1.6 mcg/kg, 1.6 mcg/kg-1.8 mcg/kg, 1.8 mcg/kg-2.0 mcg/kg, 2.0 mcg/kg-2.2 mcg/kg, 2.2 mcg/kg-2.4 mcg/kg, 2.4 mcg/kg-2.6 mcg/kg, 2.6 mcg/kg-2.8 mcg/kg, 2.8 mcg/kg-3.0 mcg/kg, 3.0 mcg/kg-3.2 mcg/kg, 3.2 mcg/kg-3.4 mcg/kg, 3.4 mcg/kg-3.6 mcg/kg, 3.6 mcg/kg-3.8 mcg/kg, 4.0 mcg/kg-4.2 mcg/kg, 4.2 mcg/kg-4.4 mcg/kg, 4.4 mcg/kg-4.6 mcg/kg, 4.6 mcg/kg-4.8 mcg/kg, 4.8 mcg/kg-5.0 mcg/kg, 5.0 mcg/kg-5.2 mcg/kg, 5.2 mcg/kg-5.4 mcg/kg, 5.4 mcg/kg-5.6 mcg/kg, 5.6 mcg/kg-5.8 mcg/kg, or 5.8 mcg/kg-6.0 mcg/kg.

In some embodiments, the RTX is administered at a dose of at least about 0.25 mcg/ml.

In some embodiments, the RTX is administered at a dose of at least about 0.3 mcg/ml.

In some embodiments, the RTX is administered at a dose of at least about 0.4 mcg/ml.

In some embodiments, the RTX is administered at a dose of at least about 0.5 mcg/ml.

In some embodiments, the RTX is administered at a dose of at least about 1.0 mcg/ml.

In some embodiments, the RTX is administered at a dose of at least about 2.0 mcg/ml.

In some embodiments, the RTX is delivered in a composition having a volume of 1 ml-10 ml, 10 ml-20 ml, 20 ml-30 ml, 30 ml-40 ml, 40 ml-50 ml, 50 ml-60 ml, 60 ml-70 ml, 70 ml-80 ml, 80 ml-90 ml, 90 ml-100 ml, 100 ml-110 ml, 110 ml-120 ml, 120 ml-130 ml, 130 ml-140 ml, 140 ml-150 ml, 160 ml-170 ml, 170 ml-180 ml, 180 ml-190 ml, 190 ml-200 ml, 200 ml-210 ml, 210 ml-220 ml, 220 ml-230 ml, 230 ml-240 ml, 240 ml-250 ml, 260 ml-270 ml, 270 ml-280 ml, 280 ml-290 ml, or 290 ml-300 ml. In some embodiments, the RTX is delivered in a composition having a volume of 5 ml-15 ml.

The dosage and volume can be adjusted depending on the size of the subject and/or the internal volume of the subject's bladder. Notably, RTX is specific for the TRPV1 receptor and therefore affects non-target nerves that do not have enough TRPV1 receptors to be sensitive to RTX to a much lesser extent. In some embodiments, the RTX is administered at a concentration and volume such that the intravesical concentration of RTX in the bladder is at least about 0.1 mcg/ml, such as 0.1 mcg/ml-0.2 mcg/ml, 0.2 mcg/ml-0.3 mcg/ml, 0.3 mcg/ml-0.4 mcg/ml, 0.4 mcg/ml-0.5 mcg/ml, 0.5 mcg/ml-0.6 mcg/ml, 0.6 mcg/ml-0.7 mcg/ml, 0.7 mcg/ml-0.8 mcg/ml, 0.8 mcg/ml-0.9 mcg/ml, or 0.9 mcg/ml-1 mcg/ml. In some embodiments, the RTX is administered at a concentration and volume such that the intravesical concentration of RTX in the bladder is at least about 1 mcg/ml, e.g., 1 mcg/ml-1.1 mcg/ml, 1.1 mcg/ml-1.2 mcg/ml, 1.2 mcg/ml-1.3 mcg/ml, 1.3 mcg/ml-1.4 mcg/ml, 1.4 mcg/ml-1.5 mcg/ml, 1.5 mcg/ml-1.6 mcg/ml, 1.6 mcg/ml-1.7 mcg/ml, 1.7 mcg/ml-1.8 mcg/ml, 1.8 mcg/ml-1.9 mcg/ml, or 1.9 mcg/ml-2 mcg/ml.

### 2. Formulations

Multiple examples of formulations of RTX are available in the literature. See, e.g., Ueda et al. (2008) J. of Cardiovasc. Pharmacol. 51:513-520, and US 2015/0190509 A1. Any suitable formulation of RTX for intravesical administration may be used. In some embodiments, RTX is prepared for administration by dilution in saline.

In some embodiments, the RTX, which may be at the dosages discussed above, is administered with a pharmaceutically acceptable carrier. In some embodiments, the pharmaceutically acceptable carrier comprises water. In some embodiments, the pharmaceutically acceptable carrier comprises saline. In some embodiments, the pharmaceutically acceptable carrier comprises polysorbate 80. In some embodiments, the pharmaceutically acceptable carrier comprises polyethylene glycol. In some embodiments, the pharmaceutically acceptable carrier comprises sugar or sugar alcohol. In some embodiments, the pharmaceutically acceptable carrier comprises mannitol. In some embodiments, the pharmaceutically acceptable carrier comprises dextrose. In some embodiments, the pharmaceutically acceptable carrier comprises a pharmaceutically acceptable buffer. In some embodiments, the pharmaceutically acceptable carrier comprises a phosphate buffer. In some embodiments, the pharmaceutically acceptable carrier comprises a pharmaceutically acceptable salt. In some embodiments, the pharmaceutically acceptable carrier comprises NaCl. In some embodiments, the pharmaceutically acceptable carrier comprises an organic solvent such as ethanol or DMSO, e.g., as a minority or residual component used as an aid in dissolving RTX before dilution in a primarily aqueous composition.

The concentration of RTX in the formulation may be any suitable value for delivery of the intended dose. In some embodiments, the concentration of RTX in the pharmaceutical formulation is in the range of 0.1 to 300 mcg/ml. In some embodiments, the concentration of RTX in the pharmaceutical formulation is in the range of 0.1-1 mcg/ml, 1-5 mcg/ml, 5-10 mcg/ml, 10-20 mcg/ml, 10-30 mcg/ml, 20-30 mcg/ml, 20-50 mcg/ml, 50-100 mcg/ml, 100-150 mcg/ml, 150-200 mcg/ml, 200-250 mcg/ml, or 250-300 mcg/ml. In some embodiments, the concentration of RTX in the pharmaceutical formulation is 0.1 mcg/ml-0.2 mcg/ml, 0.2 mcg/ml-0.3 mcg/ml, 0.3 mcg/ml-0.4 mcg/ml, 0.4 mcg/ml-0.5 mcg/ml, 0.5 mcg/ml-0.6 mcg/ml, 0.6 mcg/ml-0.7 mcg/ml, 0.7 mcg/ml-0.8 mcg/ml, 0.8 mcg/ml-0.9 mcg/ml, 0.9 mcg/ml-1.0 mcg/ml, 1.0 mcg/ml-1.1 mcg/ml, 1.1 mcg/ml-1.2 mcg/ml, 1.2 mcg/ml-1.3 mcg/ml, 1.3 mcg/ml-1.4 mcg/ml, 1.4 mcg/ml-1.5 mcg/ml, 1.5 mcg/ml-2 mcg/ml, 2 mcg/ml-3 mcg/ml, 3 mcg/ml-4 mcg/ml, 4 mcg/ml-5 mcg/ml, 5 mcg/ml-6 mcg/ml, 6 mcg/ml-7 mcg/ml, 7 mcg/ml-8 mcg/ml, 8 mcg/ml-9 mcg/ml, 9 mcg/ml-10 mcg/ml, 10 mcg/ml-11 mcg/ml, 11 mcg/ml-12 mcg/ml, 12 mcg/ml-13 mcg/ml, 13 mcg/ml-14 mcg/ml, or 14 mcg/ml-15 mcg/ml.

The formulation may have any pH suitable for intravesical administration. In some embodiments, the pharmaceutical formulation comprising the RTX and a pharmaceutically acceptable carrier has a pH in the range of 6 to 7.6. In some embodiments, the pharmaceutical formulation comprising the RTX and a pharmaceutically acceptable carrier has a pH in the range of 6 to 6.4, 6.3 to 6.7, 6.4 to 6.8, 6.8 to 7.2, 7 to 7.4, or 7.2 to 7.6. In some embodiments, the pharmaceutical formulation comprising the RTX and a pharmaceutically acceptable carrier has a pH of 6.5 or 7.2.

In some embodiments, the formulation comprises polysorbate 80. In some embodiments, the concentration of polysorbate 80 is 0.03-7% w/v. In some embodiments, the concentration of polysorbate 80 is 2-4% w/v. In some embodiments, the concentration of polysorbate 80 is 3% w/v. The formulation may further comprise a buffer, such as phosphate buffer (e.g., sodium phosphate buffer). In some embodiments, the concentration of phosphate buffer is 10-50 mM. In some embodiments, the concentration of phosphate buffer is 10-30 mM. In some embodiments, the concentration of phosphate buffer is 10 mM. In some embodiments, the concentration of phosphate buffer is 30 mM. The formulation may have a pH in the range of 7-7.5, such as about 7.2. In some embodiments, in any of the foregoing formulations, the concentration of RTX may be 10-30 mcg/ml, such as 10 mcg/ml or 25 mcg/ml. In some embodiments, the formulation further comprises phosphate buffer, e.g., at a concentration and pH shown for phosphate buffer in Table 1. In some embodiments, the formulation further comprises NaCl, e.g., at a concentration shown for NaCl in Table 1. When both are present, the phosphate buffer and NaCl may be (but are not necessarily) present at a combination of concentrations and phosphate buffer pH shown for an individual formulation.

Exemplary formulations of RTX are shown in the following table.

TABLE 1
Exemplary RTX Solution Formulations

| Formu- | | | |
|---|---|---|---|
| lation | | Component | |
| Number | Formulation Components | Concentration | |
| 1 | RTX | 200 | mcg/mL |
| | Polysorbate 80 | 7.0% | w/v |
| | Dextrose | 0.8% | w/v |
| | 30 mM Phosphate Buffer w/0.44% NaCl | 30 mM, pH 7.2 | |
| 2 | RTX | 200 | mcg/mL |
| | Polyethylene Glycol 300 | 3.0% | v/v |
| | Polysorbate 80 | 0.1% | w/v |
| | Dextrose | 0.8% | w/v |
| | 10 mM Phosphate Buffer w/0.73% NaCl | 10 mM, pH 6.5 | |
| 3 | RTX | 200 | mcg/mL |
| | Polyethylene Glycol 300 | 30.0% | v/v |
| | Polysorbate 80 | 1.0% | w/v |
| | 10 mM Phosphate Buffer w/0.86% NaCl | 10 mM, pH 6.5 | |
| 4 | RTX | 200 | mcg/mL |
| | Polyethylene Glycol 300 | 30.0% | v/v |
| | Polysorbate 80 | 0.04% | w/v |
| | 10 mM Phosphate Buffer w/0.88% NaCl | 10 mM, pH 6.5 | |
| 5 | RTX | 200 | mcg/mL |
| | Polysorbate 80 | 3.0% | w/v |
| | Dextrose | 0.8% | w/v |
| | 30 mM Phosphate Buffer w/0.54% NaCl | 30 mM, pH 7.2 | |
| 6 | RTX | 200 | mcg/mL |
| | Polysorbate 80 | 3.0% | w/v |
| | Mannitol | 0.8% | w/v |
| | 30 mM Phosphate Buffer w/0.54% NaCl | 30 mM, pH 7.2 | |
| 7 | RTX | 200 | mcg/mL |
| | Polysorbate 80 | 7.0% | w/v |
| | Mannitol | 0.8% | w/v |
| | 30 mM Phosphate Buffer w/0.45% NaCl | 30 mM, pH 7.2 | |
| 8 | RTX | 200 | mcg/mL |
| | Polyethylene Glycol 300 | 3.0% | v/v |
| | Polysorbate 80 | 0.1% | w/v |
| | Mannitol | 0.8% | w/v |
| | 10 mM Phosphate Buffer w/0.74% NaCl | 10 mM, pH 6.5 | |
| 9 | RTX | 200 | mcg/mL |
| | Polyethylene Glycol 300 | 3.0% | v/v |
| | Polysorbate 80 | 0.1% | w/v |
| | Dextrose | 3.0% | w/v |
| | 10 mM Phosphate Buffer w/0.34% NaCl | 10 mM, pH 6.5 | |
| 10 | RTX | 200 | mcg/mL |
| | Polyethylene Glycol 300 | 3.0% | v/v |
| | Polysorbate 80 | 0.1% | w/v |
| | Mannitol | 3.0% | w/v |
| | 10 mM Phosphate Buffer w/0.36% NaCl | 10 mM, pH 6.5 | |
| 11 | RTX | 200 | mcg/mL |
| | Polysorbate 80 | 0.03% | w/v |
| | Dextrose | 0.05% | w/v |
| | 30 mM Phosphate Buffer w/0.54% NaCl | 30 mM, pH 7.2 | |
| 12 | RTX | 200 | mcg/mL |
| | Polysorbate 80 | 3.0% | w/v |
| | Dextrose | 5.0% | w/v |
| | 30 mM Phosphate Buffer w/0.54% NaCl | 30 mM, pH 7.2 | |
| 13 | RTX | 25 | mcg/mL |
| | Polysorbate 80 | 3.0% | w/v |
| | Dextrose | 5.0% | w/v |
| | 30 mM Phosphate Buffer w/0.54% NaCl | 30 mM, pH 7.2 | |
| 14 | RTX | 25 | mcg/mL |
| | Polysorbate 80 | 0.03% | w/v |
| | Dextrose | 0.05% | w/v |
| | 30 mM Phosphate Buffer w/0.54% NaCl | 30 mM, pH 7.2 | |
| 15 | RTX | 100 | mcg/mL |
| | Polysorbate 80 | 0.03% | w/v |
| | Dextrose | 0.05% | w/v |
| | 30 mM Phosphate Buffer w/0.54% NaCl | 30 mM, pH 7.2 | |
| 16 | RTX | 200 | mcg/mL |
| | Polysorbate 80 | 7.0% | w/v |
| | Dextrose | 5.0% | w/v |

| | |
|---|---|
| 30 mM Phosphate Buffer w/0.54% NaCl | 30 mM, pH 7.2 |

In some embodiments, formulations in Table 1 include dextrose. In embodiments, the concentration of dextrose is 0.05-5% w/v. In some embodiments, the concentration of dextrose is 0.8-5% w/v. In some embodiments, the concentration of dextrose is 0.05% w/v. In some embodiments, the concentration of dextrose is 0.8% w/v. In some embodiments, the concentration of dextrose is 3.0% w/v. In some embodiments, the concentration of dextrose is 5.0% w/v.

In some embodiments, formulations in Table 1 include mannitol. In some embodiments, the concentration of mannitol is 0.8-3.0% w/v. In some embodiments, the concentration of mannitol is 0.8% w/v. In some embodiments, the concentration of mannitol is 3.0% w/v.

In some embodiments, the dextrose or mannitol is omitted from a formulation shown in Table 1.

In some embodiments, the concentration of RTX in a formulation shown in Table 1 is adjusted to any of the RTX concentrations or concentration ranges disclosed herein. For example, in some embodiments, the concentration of RTX in a formulation shown in Table 1 is adjusted to 0.3-200 mcg/ml. In some embodiments, the concentration of RTX in a formulation shown in Table 1 is 200 mcg/ml. In some embodiments, the concentration of RTX in a formulation shown in Table 1 is 0.3-100 mcg/ml. In some embodiments, the concentration of RTX in a formulation shown in Table 1 is 100 mcg/ml. In some embodiments, the concentration of RTX in a formulation shown in Table 1 is adjusted to 0.3-50 mcg/ml. In some embodiments, the concentration of RTX in a formulation shown in Table 1 is 25 mcg/ml. As another example, in some embodiments, the concentration of RTX in a formulation shown in Table 1 is adjusted to 0.3-15 mcg/ml. As another example, in some embodiments, the concentration of RTX in a formulation shown in Table 1 is adjusted to 0.5-10 mcg/ml. As another example, in some embodiments, the concentration of RTX in a formulation shown in Table 1 is adjusted to 0.6-1.5 mcg/ml. The dextrose or mannitol is omitted from any such formulation having an adjusted RTX concentration.

The formulations in Table 1 may be prepared according to the following exemplary methods, which are provided for formulations 3 and 5 but may be adapted to the other formulations by one skilled in the art. Formulation 3 may be made by adding 46 mg sodium phosphate monobasic monohydrate, 94.7 mg sodium phosphate dibasic anhydrous, and 860 mg NaCl to a 100 ml volumetric flask. 50 ml of water for injection (WFI) is added to dissolve the components in the flask, followed by addition of 1.0 g of polysorbate 80, to form the aqueous component. 20 mg of RTX is added to the aqueous component in the volumetric flask, and pH is adjusted with hydrochloric acid/sodium hydroxide to 7.2. Then 30 mL of PEG 300 is added and the solution is sonicated to dissolve the solids. It should be noted that RTX will sometimes precipitate at the interface of aqueous solution and PEG initially, but will go back into solution upon sonication. The full mixture in the flask is diluted to volume (100.00 ml) with water (WFI) and this is mixed by an inversion process. The full formulation is filtered through a 0.2 μm polytetrafluoroethylene (PTFE) filter.

Formulation 5 may be made by preparing adding 138 mg sodium phosphate monobasic monohydrate, 284.1 mg sodium phosphate dibasic anhydrous, and 540 mg NaCl to a 100 ml volumetric flask. 50 ml of water for injection (WFI) is added to dissolve the components in the flask, followed by addition of 3.0 g of polysorbate 80, and 800 mg of dextrose to form the aqueous component. 20 mg of RTX is added to the aqueous component in the volumetric flask, and pH is adjusted with hydrochloric acid/sodium hydroxide to 7.2. The solution is then sonicated to dissolve all the solids. (Alternatively, the RTX may be initially dissolved in a small volume of ethanol or DMSO, and this solution may then be added to the aqueous component.) The full mixture in the flask is diluted to volume (100.00 ml) with water (WFI) and this is mixed by an inversion process. The full formulation is filtered through a 0.2 μm PTFE filter.

A formulation according to Formulation 11 is prepared using 200 mcg RTX, 300 mcg Polysorbate 80 (using commercially-available polysorbate 80); 5.4 mg of sodium chloride, 500 mcg of dextrose, 1.38 mg sodium phosphate monobasic monohydrate, 2.84 mg sodium phosphate dibasic anhydrous, and water (WFI) to 1 mL, then pH is adjusted with hydrochloric acid/sodium hydroxide to 7.2. As noted above, the dextrose may be omitted.

A formulation according to Formulation 13 is prepared using 25 mcg RTX, 30 mg Polysorbate 80 (using commercially-available polysorbate 80); 5.4 mg of sodium chloride, 50 mg of dextrose, 1.38 mg sodium phosphate monobasic monohydrate, 2.84 mg sodium phosphate dibasic anhydrous, water (WFI) to 1 mL, then pH is adjusted with hydrochloric acid/sodium hydroxide to 7.2. As noted above, the dextrose may be omitted.

Further details on techniques for formulation and administration may be found in Gennaro, A., Ed., Remington's Pharmaceutical Sciences, 18th Ed. (1990) (Mack Publishing Co., Easton, Pa.).

### V. EXAMPLES

### A. Intravesical Instillation of RTX Ablates Afferent Nerves in Healthy Feline Bladders

To examine the effect of intravesical instillation of RTX on the bladder, 6 healthy cats, between the 2-5 years of age, were treated with either 5 (low dose), 25 (mid dose) or 50 mcg (high dose) of RTX (volume was 25 ml; concentrations were 0.2, 1, and 2 mcg/ml). These doses are considered equivalent to administration of 20, 100, or 200 mcg in a human with a volume of 100 ml. A seventh cat received saline only and served as the control. The bladders of the anesthetized cats were emptied and then flushed with sterile saline via a foley urinary catheter. Once the saline was removed, the treatment with RTX (test cats) or saline alone (control cat) was administered over 60 seconds and left in the bladder for 20 minutes.

The bladder of each cat was emptied and blood samples for pharmacokinetic analysis were collected pre-RTX treatment and at 0.5, 1, and 4 hours post RTX treatment. Plasma concentrations of RTX were measured using HPLC and the results are shown in Table 2 below.

TABLE 2
Plasma concentration of RTX following treatment with RTX
Plasma concentrations (pg/ml) for RTX in cats following instillation of RTX

| Time | Saline | 5 mcg total | | 25 mcg total | | 50 mcg total | |
|---|---|---|---|---|---|---|---|
| (hour) | KPX6 | JFD8 | IOS7 | JIB5 | JIR6 | KQD5 | JIL6 |
| 0 | BQL | BQL | BQL | BQL | BQL | BQL | BQL |
| 0.5 | BQL | BQL | BQL | 55.5 | 10.1* | 8.77* | BQL |
| 1 | BQL | BQL | BQL | 13.5* | BQL | BQL | BQL |
| 4 | — | BQL | BQL | BQL | BQL | BQL | BQL |

*Sample contained adequate signal-to-noise; value reported as an estimate.
BQL = Below the Quantifiable Limit (<50.0 pg/mL).

Plasma levels of RTX were below 50 pg/mL in all 3 groups at all time points (0, 0.5, 1 and 4 h), except at 0.5 h for 25 mcg and 50 mcg groups (with peak values ranging from 8.77 to 55.5 pg/mL) observed at 0.5 hour, and 1 h for the 25 mcg group. At 4 hours, RTX in plasma was below quantifiable levels in all groups.

A physical exam, complete blood count and chemistry parameters were performed on day 0, 7, and 14. Complete blood count and chemistry parameters were analyzed at the Clinical Pathology Laboratory at the University of Georgia. Food consumption and signs of abnormal urination were monitored through the study period. RTX was well tolerated with no clinical adverse effects noted during or post administration at all doses tested (5 mcg, 25 mcg, or 50 mcg).

At day 14 the cats were anesthetized and perfused with saline followed by 10% formalin. After confirmation of successful euthanasia, lumbar and sacral segments of the spinal cord with corresponding dorsal spinal ganglia, the urinary bladder and urethra were harvested and tissues were prepared for immunohistochemistry.

Previous studies have shown that TRPV1-immunoreactive axons co-express Calcitonin gene-related peptide (CGRP) and substance P (SP) (See, e.g., Sharrad et al. (2015) Neurosci. Lett., 599: 164-171; Bae et al. (2004) J. Comp. Neurol. 478: 62-71). Since feline bladder connective tissue, smooth muscle, and cell bodies of S2 DRG express CGRP+ and SP+ axons, expression of CGRP and SP were used in these studies to identify the majority of TRPV1 axons innervating the cat bladder and TRPV1+ cell bodies in cat S2 dorsal root ganglia (DRG).

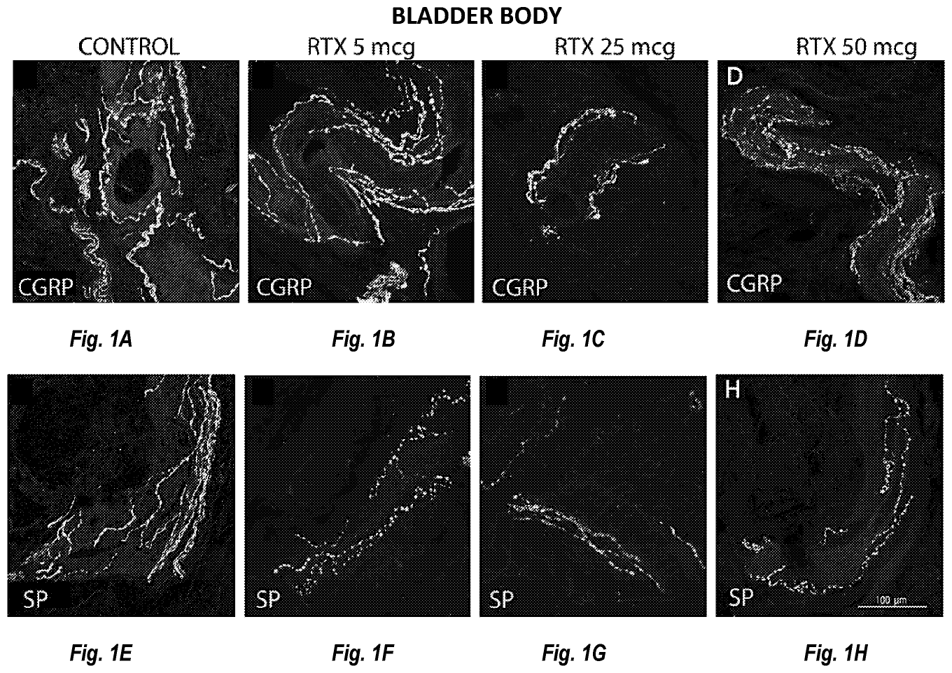

FIGS. 1A-1H show representative confocal images demonstrating the presence of CGRP (FIGS. 1B, 1C, and 1D) and SP (FIGS. 1F, 1G and 1H) axons innervating cat bladder body after the single administration of RTX at doses of 5, 25 and 50 mcg. The density of both markers was reduced in cats treated with RTX (14 days post-treatment) when compared to control (saline). These results indicate that there is a reduction of both CGRP and SP axons innervating the bladder body.

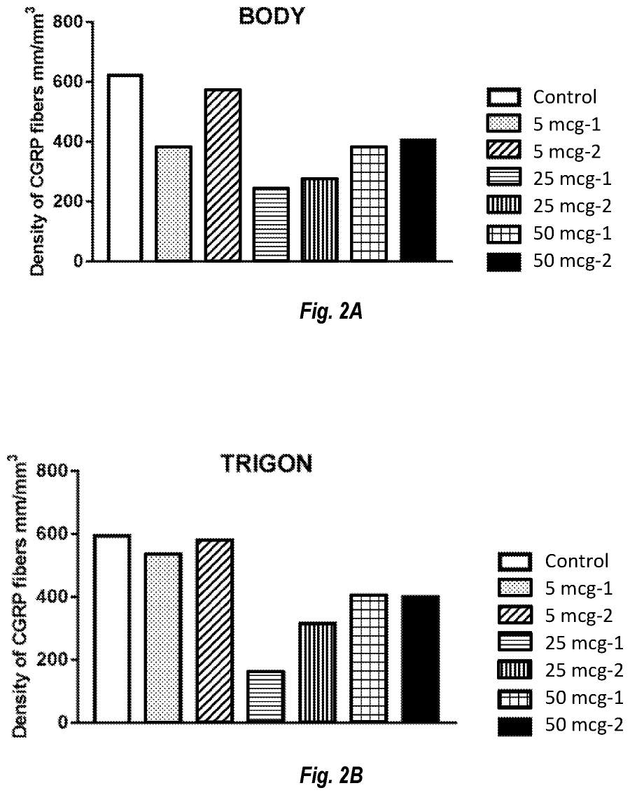

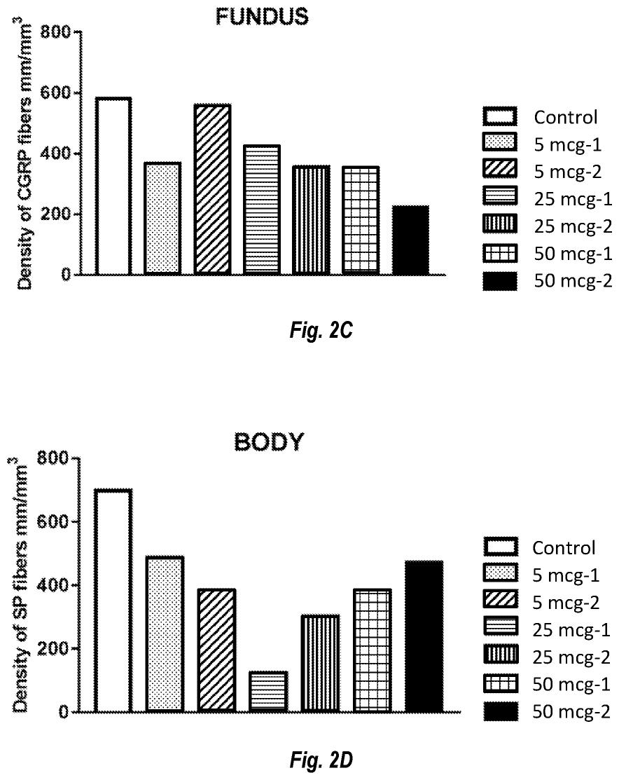

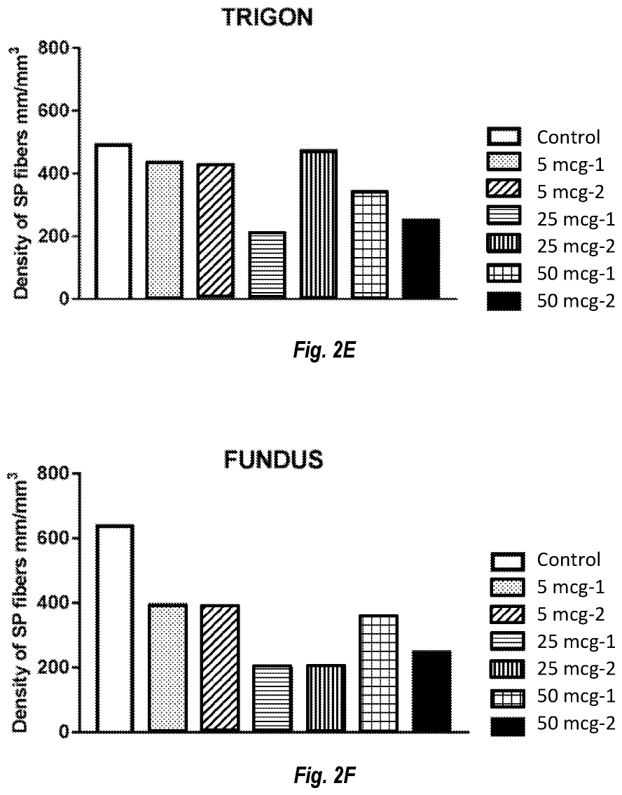

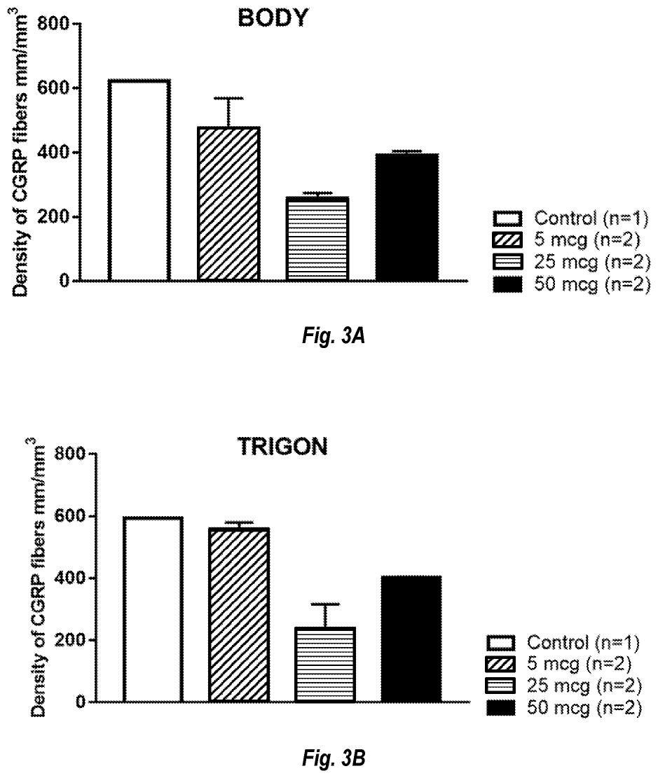

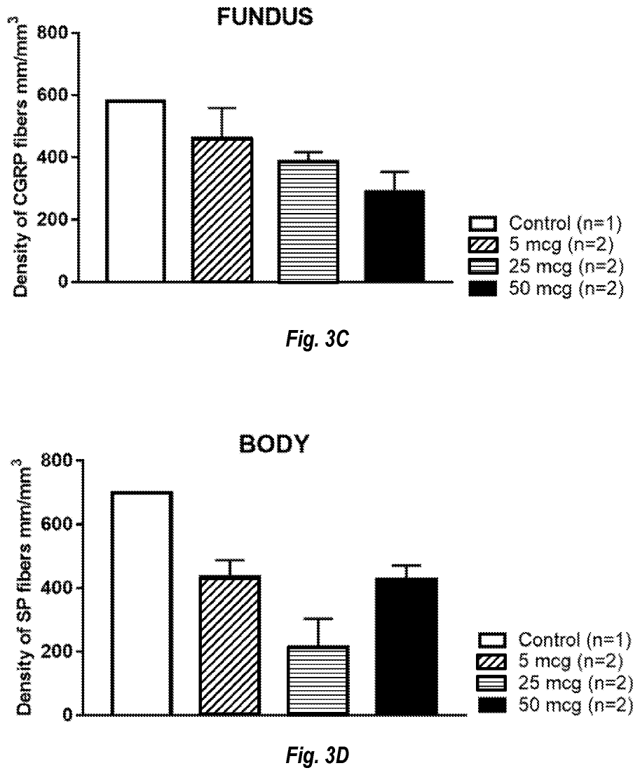

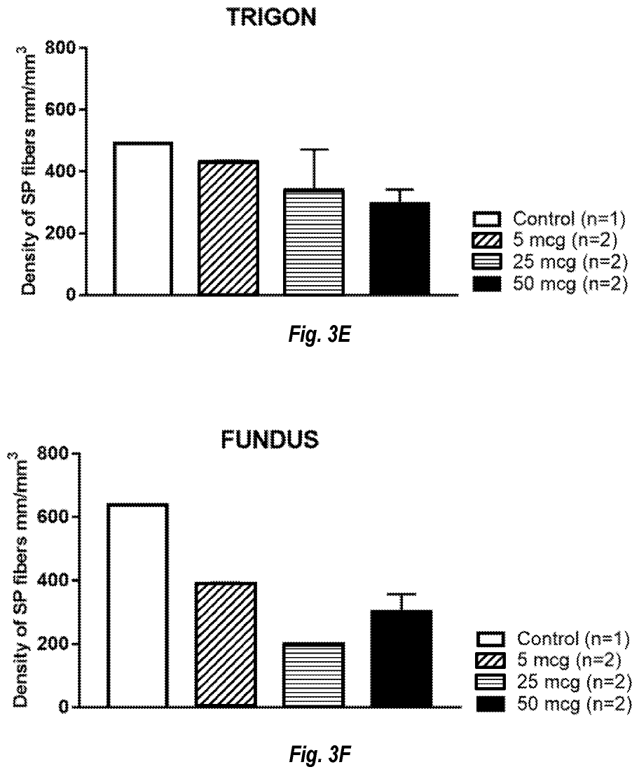

FIGS. 2A-2F show the density of CGRP and SP afferent nerve fibers in the body, trigon and fundus of the bladder of each individual cat. FIGS. 3A-3F show the mean density of CGRP and SP afferent nerve fibers in the body, trigon and fundus of the bladder. Cats treated with 5 mcg RTX in 25 ml of saline generally showed a modest reduction in the density of CGRP or SP fibers. Cats in the 25 or 50 mcg in 25 ml of saline treatment groups showed a more significant reduction in density of CGRP and SP fibers in the body, trigon and fundus when compared to the control. The data indicate that 25 mcg and 50 mcg doses of RTX in 25 ml of saline are effective at ablating afferent nerves in that procedure.

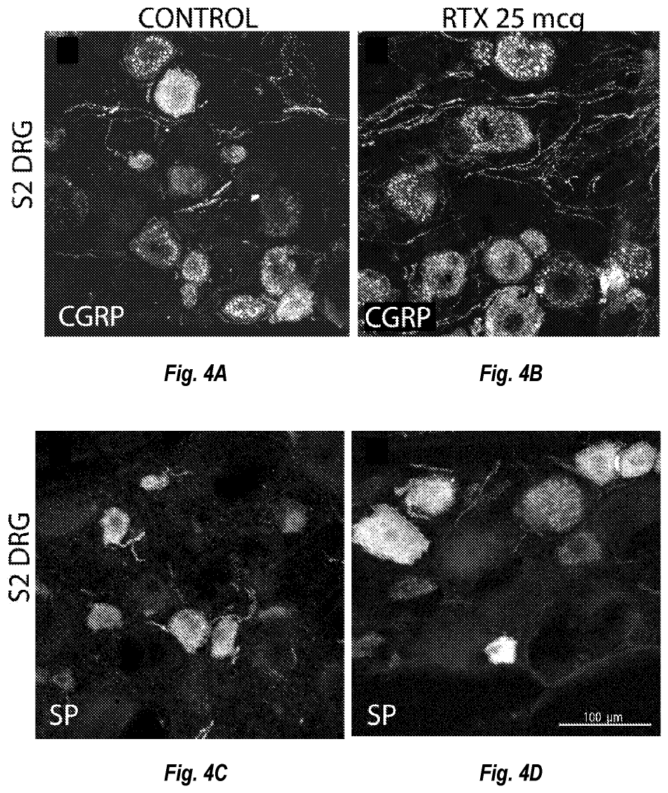

The percent of cell bodies from S1 and S2 DRG expressing SP and CGRP from feline bladders after intravesical application of either control (saline) or RTX (5, 25 or 50 mcg) was also examined using immunohistochemistry and confocal laser microscopy as described above. In both S1 and S2 DRG samples, CGRP immunostaining was observed in the cell bodies and their axons. CGRP positive cell bodies were small to medium-size. Quantitative immunohistochemical analysis for SP in DRG revealed that this neuropeptide is expressed in fewer neurons when compared to CGRP, and its expression is restricted to small to medium-size primary afferent neurons. As shown in FIG. 4A-4D, confocal images of S2 DRGs from cats treated with saline control (FIGS. 4A and 4C) or intravesical RTX at 25 mcg (FIGS. 4B and 4D) show that the number of cell bodies expressing CGRP and SP does not decrease after RTX treatment.

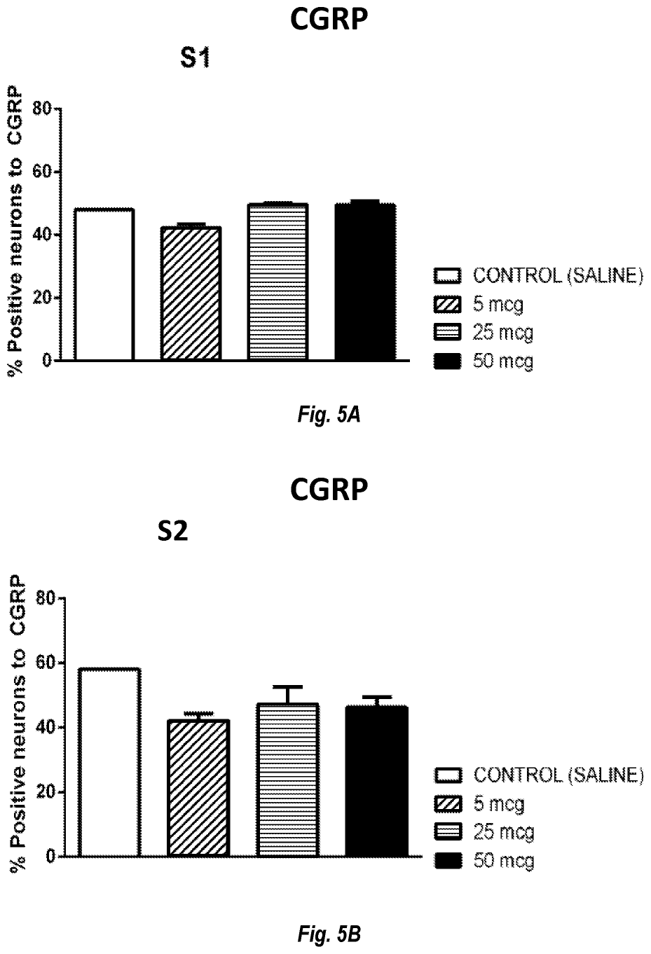

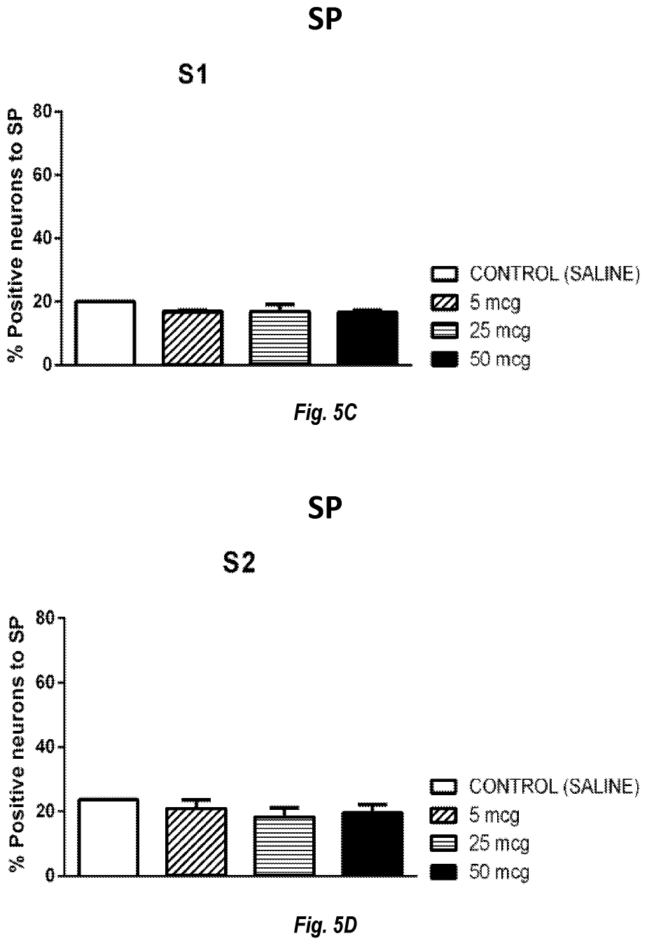

FIGS. 5A-5D show that that intravesical RTX treatment at doses of 5, 25, or 50 mcg did not affect the percentage of cell bodies expressing CGRP and SP in the S1 and S2 DRG when compared to control. The percentage of CRGP positive cells in the S1 and S2 DRG is shown in FIGS. 5A and 5B, and the percentage of SP positive cells in the S1 and S2 DRG is shown in FIGS. 5C and 5D, respectively.

### B. Pharmacokinetic and Toxicokinetic Profile of RTX Following Intravesical Instillation in Dog Bladder

Local tolerance and the pharmacokinetic (PK), and toxicokinetic (TK) profile of RTX in dogs was determined after a single instillation of RTX into the bladder of dogs (n=6). The RTX formulations were prepared using aseptic techniques in a biological safety cabinet (BSC) by mixing the appropriate amount of diluent with the appropriate amount of RTX. Dosing formulation concentrations used were 1 and 10 mcg/mL.

Three male and three female dogs ranging in weight from 5.95 to 8.60 kg were used in the study. The dogs were assigned into Group 1 (receiving a dose of 1 mcg/mL RTX) or Group 2 (receiving a dose of 10 mcg/mL RTX) as shown in the Table 3 below.

TABLE 3
Experimental Design

| | | Dosage | Dose | | |
|---|---|---|---|---|---|
| Group | RTX Dose | Volume^{a} | Concentration | No. of Animals^{b} | |
| No. | (mcg) | (mL) | (mcg/mL) | Males | Females |
| 1 | 25 | 25 | 1 | 1 | 1 |
| 2 | 250 | 25 | 10 | 2 | 2 |

^{a}The route of administration for both groups was intravesical instillation into the urinary bladder.
^{b}The animals were maintained until Day 8.

RTX was administered via intravesical instillation into the urinary bladder. The animals were given a single dose of 25 or 250 mcg/dose in a total volume of 25 mL on Day 1 over a period of 1 hour (±5 minutes). RTX and diluent were administered to the dogs under general anesthesia via a Foley catheter placed into the bladder for 1 hour (±5 minutes).

Observations for morbidity, mortality, injury, and the availability of food and water were conducted twice daily for all animals. Observations for clinical signs for all animals were conducted on Day 2. The observations included evaluation of the skin, fur, eyes, ears, nose, oral cavity, thorax, abdomen, external genitalia, limbs and feet, respiratory and circulatory effects, autonomic effects such as salivation, and nervous system effects including tremors, convulsions, reactivity to handling, and unusual behavior.

Blood samples were collected from all animals on Day 1 pre-dose and at 0.5, 1, 2, 4, 6, 12, 24, 36, 48, 60, and 72 hours post-dose. The toxicokinetic (TK) parameters were determined for RTX, and if appropriate, the metabolites, from mean concentration-time data.

The plasma concentrations (pg/mL) for RTX in Group 1 dogs following a 25 mL intravesical administration of RTX at 1 mcg/mL (total 25 mcg/dose) were below the quantifiable limit (<50.0 pg/mL) at 2 hours post-dosing in both the male and female dog. Results are shown below in Table 4. Individual, mean, SD, and % CV values of the plasma concentrations and the TK parameters of RTX in dogs following a 250 mcg dose (Group 2) are presented in Table 5 (males) and Table 6 (females). Following a single intravesical instillation into the urinary bladder of RTX at 250 mcg to male and female dogs, RTX was absorbed rapidly into the systemic circulation. The highest RTX concentrations were observed at a Tmax value of 0.75±0.354 hours in both males and females and fell to zero by 4 hours post-dose. In males, RTX Cmax value was 352±124 pg/mL and the AUC(0-T) value was 361±6.36 pg·h/mL as shown in Table 5.

TABLE 4
Plasma concentrations of dogs treated with a 25 mcg dose of RTX.

| | Male Dog. | Female Dog. |
|---|---|---|
| | I.D. | I.D. |
| Time (h) | 1001 | 1002 |
| Predose | BQL | BQL |
| 0.5 | BQL | 39.2* |
| 1 | 15.5* | 20.3* |
| 2 | BQL | BQL |
| 4 | BQL | BQL |
| 6 | BQL | BQL |
| 12 | BQL | BQL |
| 24 | BQL | BQL |
| 36 | BQL | BQL |
| 48 | BQL | BQL |
| 60 | BQL | BQL |
| 72 | BQL | BQL |

*BQL—Below quantifiable limit (<50.0 pg/mL); adequate signal-to-noise enabled reporting value as estimate

TABLE 5
Plasma concentrations (pg/mL) and toxicokinetic parameters
of RTX in male dogs following a 250 mcg intravesical
instillation of RTX into the urinary bladder.

| | Male Dog | | | | |
|---|---|---|---|---|---|
| | I.D. | | | | |
| Time (h) | 1003 | 1005 | Mean | SD | % CV |
| Predose | 0.00 | 0.00 | 0.00 | 0.00 | — |
| 0.5 | 257 | 440 | 349 | 129 | 37.1 |
| 1 | 264 | 151 | 208 | 79.9 | 38.5 |
| 2 | 59.7 | 63.3 | 61.5 | 2.55 | 4.14 |
| 4 | 0.00 | 0.00 | 0.00 | 0.00 | — |
| 6 | 0.00 | 0.00 | 0.00 | 0.00 | — |
| 12 | 0.00 | 0.00 | 0.00 | 0.00 | — |
| 24 | 0.00 | 0.00 | 0.00 | 0.00 | — |
| 36 | 0.00 | 0.00 | 0.00 | 0.00 | — |
| 48 | 0.00 | 0.00 | 0.00 | 0.00 | — |
| 60 | 0.00 | 0.00 | 0.00 | 0.00 | — |
| 72 | 0.00 | 0.00 | 0.00 | 0.00 | — |
| Cmax, pg/mL | 264 | 440 | 352 | 124 | 35.2 |
| Cmax/Dose, pg/mL/ | 1.06 | 1.76 | 1.41 | 0.495 | 35.1 |
| (mcg) | | | | | |
| Tmax, h | 1.0 | 0.5 | 0.75 | 0.354 | 47.2 |
| AUC(0-T), pg · h/mL | 356 | 365 | 361 | 6.36 | 1.76 |
| AUC(0-T)/Dose, | 1.42 | 1.46 | 1.44 | 0.0254 | 1.76 |
| pg · h/mL/(mcg) | | | | | |
| Cmax Ratio (Male/ | — | — | 1.28 | — | — |
| Female) | | | | | |
| AUC(0-T) Ratio | — | — | 1.23 | — | — |
| (Male/Female) | | | | | |

Cₘₐₓ is the observed maximum plasma concentration from the start of dosing. Tₘₐₓ is the time to reach the Cₘₐₓ. AUC_{(0-T)} is the area under the plasma concentration-time curve from time zero to the last measurable time point calculated by the linear trapezoidal rule.

In females, RTX Cₘₐₓ value was 274±161 pg/mL and the AUC_{(0-T)} value was 294±209 pg·h/mL as shown in Table 6 below.

TABLE 6
Plasma concentrations (pg/mL) and toxicokinetic parameters
of RTX in female dogs following a 250 mcg intravesical
instillation of RTX into the urinary bladder.

| | Female Dog I.D. | | | | |
|---|---|---|---|---|---|
| Time (h) | 1004 | 1006 | Mean | SD | % CV |
| Predose | 0.00 | 0.00 | 0.00 | 0.00 | — |
| 0.5 | 388 | 0.00 | 194 | 274 | 141 |
| 1 | 289 | 160 | 225 | 91.2 | 40.5 |
| 2 | 60.2 | 51.1 | 55.7 | 6.43 | 11.5 |
| 4 | 0.00 | 0.00 | 0.00 | 0.00 | — |
| 6 | 0.00 | 0.00 | 0.00 | 0.00 | — |
| 12 | 0.00 | 0.00 | 0.00 | 0.00 | — |
| 24 | 0.00 | 0.00 | 0.00 | 0.00 | — |
| 36 | 0.00 | 0.00 | 0.00 | 0.00 | — |
| 48 | 0.00 | 0.00 | 0.00 | 0.00 | — |
| 60 | 0.00 | 0.00 | 0.00 | 0.00 | — |
| 72 | 0.00 | 0.00 | 0.00 | 0.00 | — |
| Cmax, pg/mL | 388 | 160 | 274 | 161 | 58.8 |
| Cmax/Dose, pg/mL/ | 1.55 | 0.640 | 1.10 | 0.643 | 58.5 |
| (mcg) | | | | | |
| Tmax, h | 0.5 | 1.0 | 0.75 | 0.354 | 47.2 |
| AUC(0-T), pg · h/mL | 441 | 146 | 294 | 209 | 71.1 |
| AUC(0-T)/Dose, | 1.76 | 0.584 | 1.18 | 0.832 | 70.5 |
| pg · h/mL/(mcg) | | | | | |

The systemic TK exposure of RTX was demonstrated in dogs when administered as a single intravesical instillation into the urinary bladder of 250 mcg to male and female dogs. The data show that RTX was absorbed rapidly into the systemic circulation with the highest RTX concentrations observed at Tₘₐₓ value of 0.75 hour in males and females. RTX exposure in males appeared to be similar to that in females RTX male/female ratios for Cₘₐₓ was 1.28 and for the AUC_{(0-T)} was 1.23.

In summary, RTX administration was well tolerated and did not result in any detailed clinical observations or changes in body weight.

### C. Intravesical Instillation of RTX Ablates Afferent Nerves in Healthy Canine Bladders

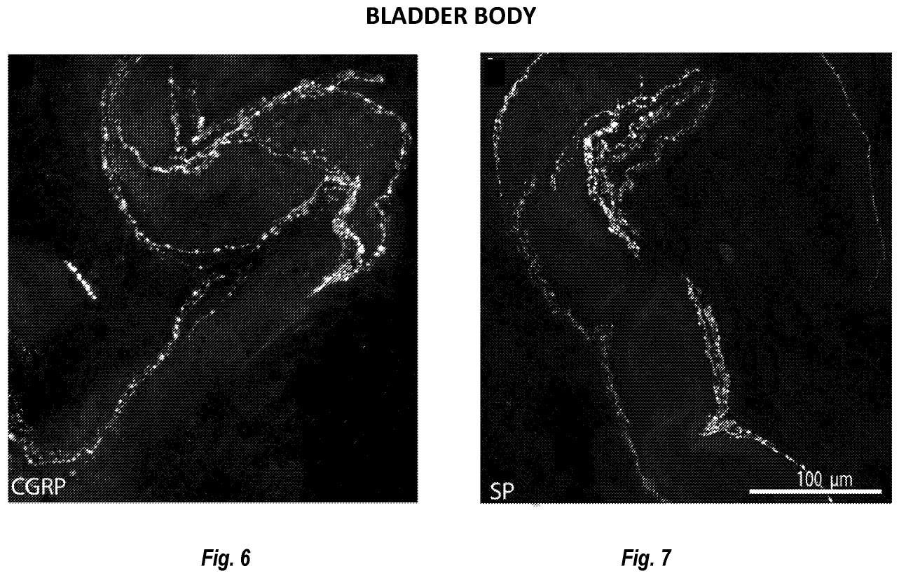

Similarly to cat bladders described in the examples above, immunohistochemical staining revealed that healthy dog bladder from an unperfused animal is also innervated by CGRP and SP positive axons present as single axons or bundles of nerve fibers as shown in FIGS. 6 and 7 respectively. Canine bladders from a healthy dog (control) and from dogs with intravesical application of resiniferatoxin (RTX) at 2 different doses (25 mcg and 250 mcg) were prepared for immunohistochemistry and confocal laser microscopy to quantify the axons expressing substance P (SP) and calcitonin-gene related peptide (CGRP). Confocal images were obtained at 40× magnification using a Carl Zeiss scanning confocal laser microscope (model LSM 800, Jena, Germany) with Z-stack function.

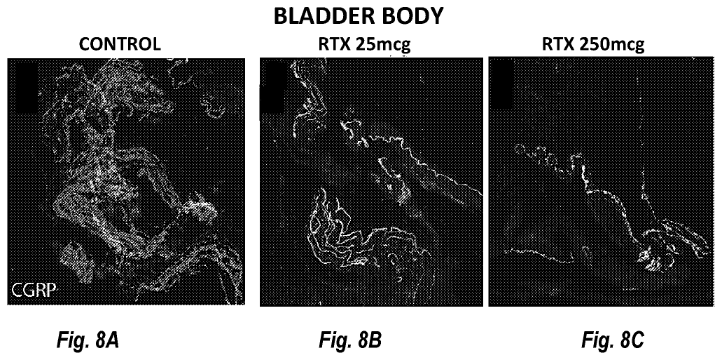

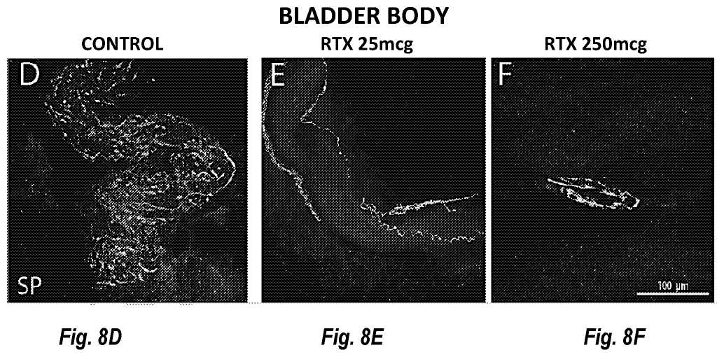

FIG. 8 shows representative confocal images which illustrate the presence of CGRP (FIGS. 8A-8C) and SP (FIGS. 8D-8F) immunoreactive axons innervating the body of the canine bladder from a healthy dog (FIGS. 8A and 8D), from animals that were treated with RTX at a dose of 25 mcg (FIGS. 8B and 8E), or 250 mcg (FIGS. 8C and 8F). Intravesical RTX treatment at doses of 25 mcg and 250 mcg resulted in a drastic reduction of/both CGRP and SP axons innervating the bladder body when compared with tissue from control dog.

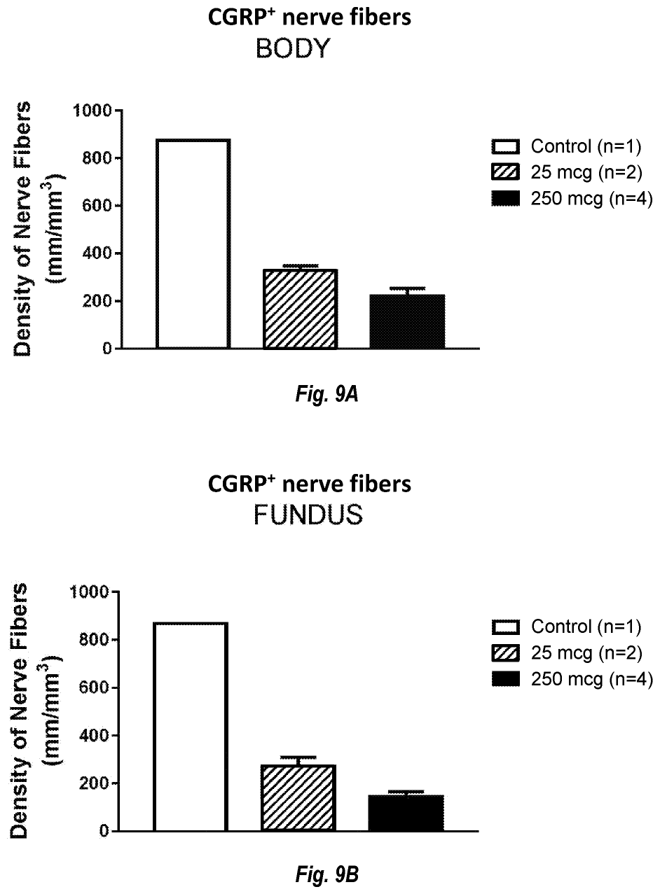

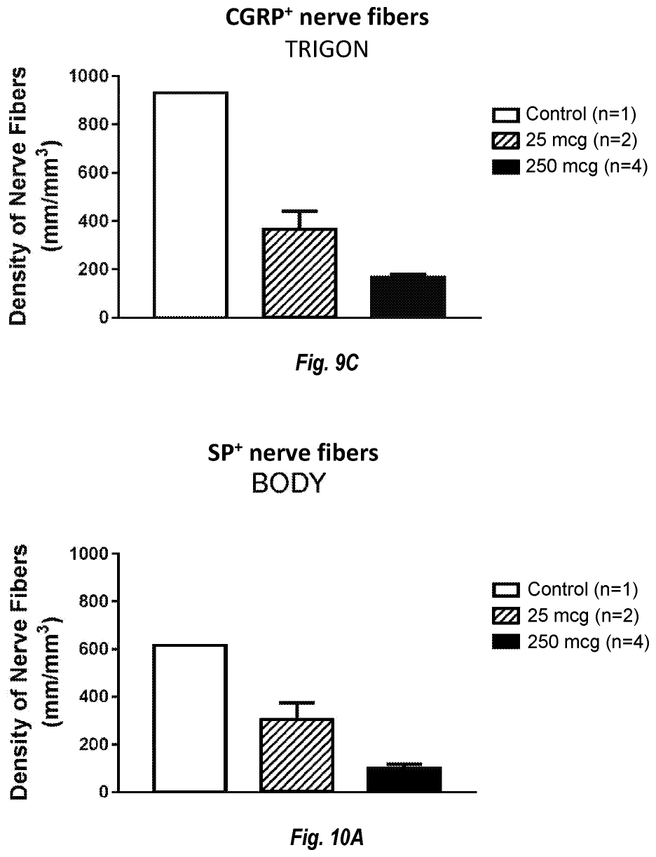

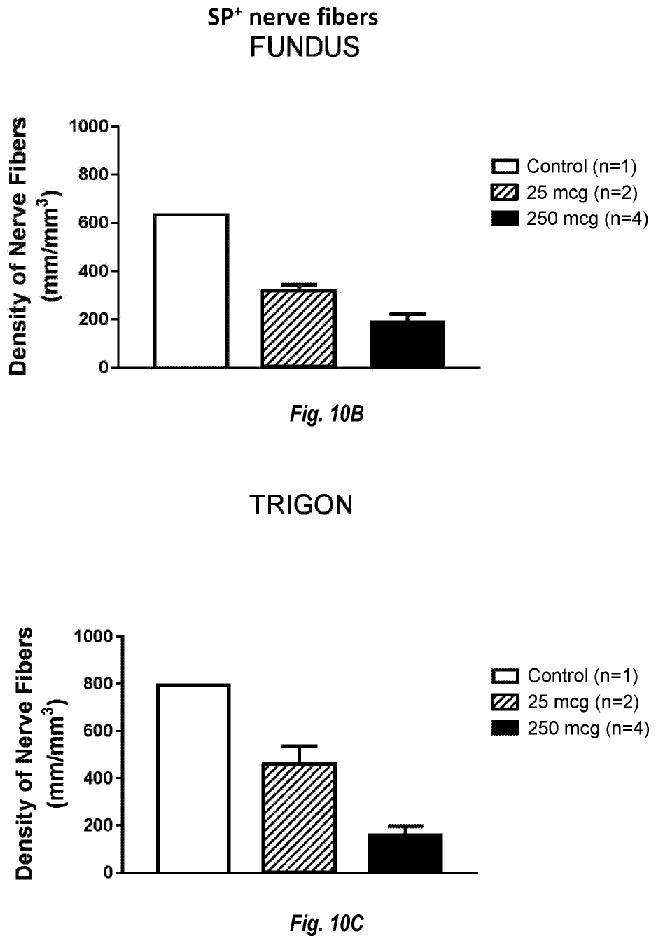

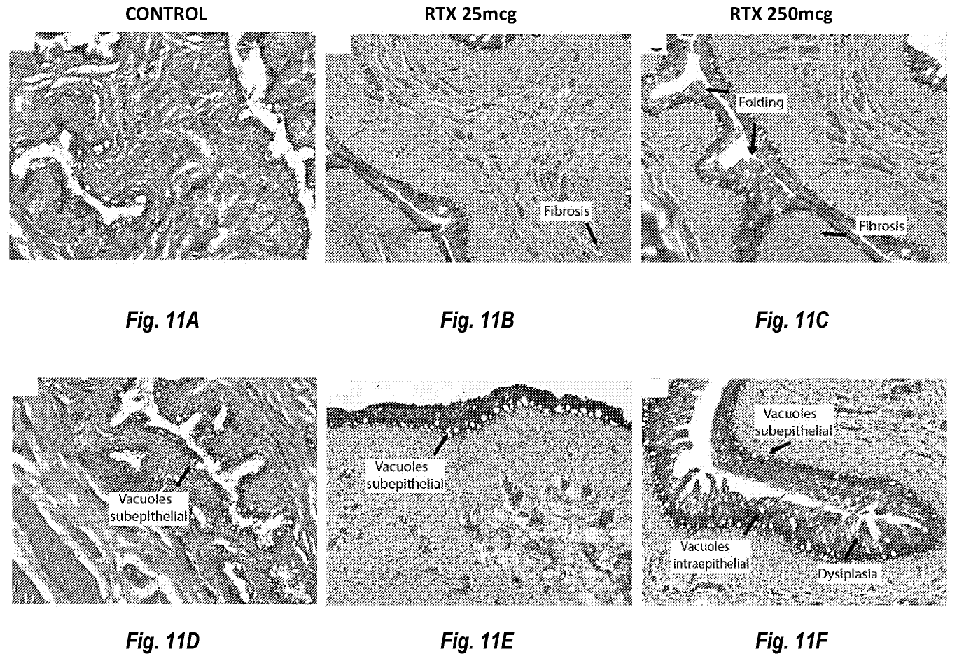

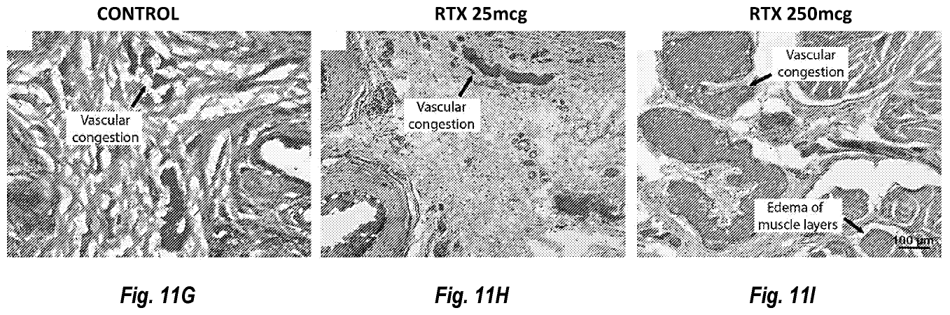

To quantify the density of SP and CGRP-immunoreactive axons in the different regions of canine bladders, the sections (n=1 for control, n=2 for 25 mcg RTX, and n=4 for 250 mcg) were observed at 10× magnification to allow the identification of the areas with the greatest density of nerve fibers. As shown in FIGS. 9A-C and 10A-C, quantitative analysis revealed that intravesical RTX treatments at both doses reduced the density of CGRP and SP nerve fibers innervating the body, trigon, and fundus compared to control tissue. The dose of 250 mcg has a greater effect in reducing the density of both CGRP and SP as compared with dogs who received 25 mcg RTX

### D. Intravesical Instillation of RTX Ablates Afferent Nerves in Diseased Bladders

Dogs with bladder cancer were treated with RTX as follows. Under general anesthesia, RTX was infused directly into the bladder using a foley catheter. RTX remained in the bladder for 30 minutes before removal.

6 dogs were enrolled and treated at the following doses:
<10 kg: RTX 25 ug diluted in 24 ml NaCl (1.0 ug/ml solution)
>10 kg: RTX 50 ug diluted in 48 ml NaCl (1.0 ug/ml solution)

7 dogs were then enrolled and treated at the following doses:
<10 kg: RTX 25 ug diluted in 11.5 ml NaCl (2.0 ug/ml solution)
10-25 kg: RTX 50 ug diluted in 23 ml NaCl (2.0 ug/ml solution)
>25 kg: RTX 100 ug diluted in 46 ml NaCl (2.0 ug/ml solution)

Three dogs were treated twice with at least 28 days between treatments. Two dogs were lost to follow-up after day 0.

To the extent adverse events occurred, they were minimal, mild, and transient.

The primary endpoint was assessment of improvement of lower urinary tract signs with a 4-point lower urinary tract symptom scale (character of urination/degree of straining; frequency of urination (daytime); frequency of urination (nighttime); and blood in urine). The secondary endpoints were anti-cancer effect, as measured by bladder ultrasound, and quality of life.

TABLE 8
Primary endpoint results.

| | Total Number | | | | | |
|---|---|---|---|---|---|---|
| | of Dogs Treated | Day 7 | Day 14 | Day 28 | Day 56 | Day 84 |
| Primary Efficacy Variable | n = 13 | n = 11 | n = 11 | n = 8 | n = 5 | n = 3 |
| Improvement of at least 1 score | | 6 (54.5%) | 8 (72.7%) | 6 (75.0%) | 5 (100%) | 3 (100%) |
| Improvement of at least 2 scores | | 4 (36.4%) | 5 (45.5%) | 4 (50.0%) | 3 (60.0%) | 2 (66.7%) |

Preliminary anti-cancer results were obtained as follows:

Case 03-01. 9.0 kg neutered male. RTX dose: 25 ug (2.8 ug/kg). TCC sum longest diameter: 6.6 cm. Adverse events considered possibly attributable to RTX: Day 0: Bladder spasm, urinating around catheter (Grade 1). Best overall tumor response: −30% change in largest tumor diameter at Day 84 (noted observation of metastatic liver nodules and regional lymphadenopathy at Day 28).

Case 41-02. 38.8 kg spayed female. RTX dose=50 ug (1.3 ug/kg), dosed twice 28 days apart. TCC sum longest diameter=3.4 cm. Adverse events considered possibly attributable to RTX: Day 1: crying out after urination (Grade 1). Best Overall Response: −24% tumor volume at Day 28 (progressive disease at Day 56 and withdrawn from study).

Case 47-02. 25.0 kg neutered male. RTX dose: 50 ug (2.0 ug/kg). TCC longest diameter: 4.4 cm. Adverse events considered possibly attributable to RTX: Day 0: diarrhea, vomiting, hyperoxia (Grade 1). Best Overall Response: N/A (no post-treatment measurements recorded; removed from study prior to Day 14 due to progressive lower urinary tract signs).

Case 03-02. 19.1 kg spayed female. RTX dose: 50 ug (2.6 ug/kg). TCC longest diameter: 2.5 cm. Adverse events considered possibly attributable to RTX: none. Best Overall Response: N/A, lost to follow-up after Day 0.

In summary, RTX was tolerated at all tested doses, including the maximum dose of 50 ug or 2.8 ug/kg dosed twice 28 days apart. All adverse events were grade 1. No dose-limiting toxicity was observed.

### E. Intravesical Instillation of RTX in Cats with Feline Idiopathic Cystitis

Cats with FIC (Feline Idiopathic Cystitis) experience constant signaling which stimulates central plasticity. Interruption of this signaling could control maladaptive pain from the bladder over the long-term by breaking the neurogenic inflammation central stimulation. To examine the effect of intravesical instillation of RTX on cats with FIC (Feline Idiopathic Cystitis), a group of cats was treated with RTX as described below.

The effectiveness and safety of a single intravesical instillation with RTX was evaluated for the management of clinical and behavioral signs associated with non-obstructive FIC in cats. The primary effectiveness endpoint was the improvement in a Veterinary-specific Outcome Measure (VSOM) and Cystitis Events Survey on day 28.

The total VSOM is the sum of three individual VSOM scores that each have an integer value in the range of 1 to 5. Each individual VSOM score corresponds to a clinical sign/activity/behavior selected as relevant to FIC for an individual cat by the cat's owner or legal representative with the help of the Investigator. An example of a clinical sign/activity/behavior is pain or resentfulness on palpation. The minimum possible score for each individual VSOM score is 1, which indicates no problem, and the maximum is 5, which indicates an impossible activity/behavior or a maximum severity clinical sign. The minimum and maximum total VSOM scores are 3 and 15. On screening days (e.g., Day 0 and Day 28), the Investigator completed the VSOM questionnaire and assigned a score to each of the 3 clinical signs/activities/behaviours that were selected.

Treatment success was defined as “a reduction of at least 2 in total VSOM score at Day 3 and 28 compared to VSOM score at day 0.” A decrease of less than 2, no change, or an increase in total score was defined as treatment failure. Cats presenting an increase in any individual VSOM score were considered a treatment failure regardless of total VSOM score.

The Cystitis Events Survey consists a 5-point scale, ranging from 0=normal cat to 5=very severe sign of cystitis. Clinical signs of cystitis included in this survey are: increased frequency of urination; straining while urinating; crying out/vocalizing while urinating; presence of blood in urine; urination outside of litter box; and increased grooming around perineum.

The use of the Cystitis Events Survey was exploratory, and no strict definition of success was applied to the results. However, and based on published literature, the results were described as follows: (1) percentage of cats per cohort with an average improvement of 2 or more points and no increase in any individual score on Day 28; and (2) percentage of cats per cohort with an average improvement of 3 or more points and no increase in any individual score on Day 28.

RTX was provided as a 25 μg/mL (2.4 mL) solution, formulated with polysorbate, glucose, and phosphate buffer, and diluted in saline to 1 μg/mL before instillation. Intravesical instillation: After the cat has reached an adequate anesthesia level, the perineal area was prepared using a standard aseptic technique. After preparation, a urinary catheter (type and size decided on a case by case basis) was placed using aseptic technique. Then, the urinary bladder was emptied and flushed with 20 ml of sterile saline. Once the flush solution was removed, the RTX solution was infused through the urinary catheter over 2 minutes. Once the total volume of the solution is infused, the solution was left in the urinary bladder for 20 min. At the end of this period the IVP (investigational veterinary product) solution was removed. Finally, the urinary catheter was removed, and the perineal area was flushed with 20 ml of saline. The total amount of solution removed from the bladder was recorded.

24 cats were treated and the cohorts and their respective IVP treatments are described in the table 9 below. The cohorts were populated and dosed sequentially. Cats enrolled in Cohorts 2, 3 and 4 were dosed at least 3 days after the previous cohort closed.

TABLE 9
Description of cohorts

| | | | | Intravesical | | |
|---|---|---|---|---|---|---|
| | | Total | | instillation | Final IVP | |
| Cohort | n | Dose | | vol. | concentration | |
| 1 | 6 | 25 | μg | 25 mL | 1 | μg/mL |
| 2 | 6 | 6.25 | μg | 25 mL | 0.25 | μg/mL |
| 3 | 6 | 12.5 | μg | 25 mL | 0.5 | μg/mL |
| 4 | 6 | 12.5 | μg | 10 mL | 1.25 | μg/mL |

The dose paradigm of Cohort 1 was selected considering good tolerability of 25 μg of RTX as an intravesical treatment, the tolerable intravesical infusion volume in an average size cat (25 mL). Dosing for Cohort 2 was selected to determine a minimal effective dose. The dose and concentration subsequently increased in Cohorts 3 and 4, respectively. This dose paradigm aimed to determine the effect of the dose versus concentration in effectiveness outcomes.

The RTX solution was provided as a 25 μg/mL (2.4 mL) solution, formulated with polysorbate, glucose, phosphate buffer, dextrose monohydrate, and sodium chloride.

Cats enrolled in Cohorts 1-4 were evaluated through 28 days and the results are summarized below—descriptive statistics for VSOM and cystitis events survey scores are shown in Tables 10 and 11, respectively.

TABLE 10
Descriptive statistics for VSOM

| Dose | Study Day | n | Mean | SD | Minimum | Median | Maximum |
|---|---|---|---|---|---|---|---|
| 25 μg | Day 0 | 6 | 10.00 | 1.095 | 9.0 | 10.00 | 12.0 |
| (1 μg/mL) | Day 28 | 5 | 1.80 | 1.643 | 0.0 | 1.00 | 4.0 |
| 6.25 μg | Day 0 | 6 | 10.00 | 1.414 | 8.0 | 10.00 | 12.0 |
| (0.25 μg/mL) | Day 28 | 6 | 6.00 | 3.162 | 1.0 | 6.50 | 10.0 |
| 12.5 μg | Day 0 | 6 | 9.67 | 1.633 | 8.0 | 9.50 | 12.0 |
| (0.5 μg/mL) | Day 28 | 5 | 1.60 | 1.517 | 0.0 | 1.00 | 4.0 |
| 12.5 μg | Day 0 | 6 | 9.83 | 0.408 | 9.0 | 10.00 | 10.0 |
| (1.25 μg/mL) | Day 28 | 6 | 3.83 | 1.602 | 2.0 | 4.00 | 6.0 |

TABLE 11
Descriptive statistics for cystitis events survey

| Dose | Study Day | n | Mean | SD | Minimum | Median | Maximum |
|---|---|---|---|---|---|---|---|
| 25 μg | Day 0 | 6 | 16.67 | 3.830 | 11.0 | 16.50 | 22.0 |
| (1 μg/mL) | Day 28 | 5 | 4.40 | 2.510 | 2.0 | 4.00 | 7.0 |
| 6.25 μg | Day 0 | 6 | 13.00 | 3.347 | 10.0 | 12.00 | 18.0 |
| (0.25 μg/mL) | Day 28 | 6 | 8.33 | 3.615 | 3.0 | 8.00 | 14.0 |
| 12.5 μg | Day 0 | 6 | 11.33 | 1.366 | 10.0 | 11.00 | 13.0 |
| (0.5 μg/mL) | Day 28 | 5 | 5.20 | 1.483 | 3.0 | 5.00 | 7.0 |
| 12.5 μg | Day 0 | 6 | 12.5 | 2.739 | 9.0 | 12.50 | 16.0 |
| (1.25 μg/mL) | Day 28 | 6 | 5.33 | 1.966 | 3.0 | 6.00 | 8.0 |

The success or failure of the VSOM treatment of cats in Cohorts 1-4 are shown below in Table 12. A total of 22 cats (22/24) were evaluated for VSOM success/failure on Day 28 compared to Day 0. One animal of Cohort 1 died after drug; necropsy was not performed. One animal of Cohort 3 was euthanized before the final evaluation due to Feline Infectious Peritonitis (FIP). Success rates were significant across cohorts, with a minimum success rate of 50% in Cohort 2 (6.25 μg/mL) and a maximum success rate of 100% in Cohorts 1 (25 μg/25 mL) and 3 (12.5 μg/25 mL).

TABLE 12
VSOM treatment success by cohort

| Success/ | | Cohort 1 | Cohort 2 | Cohort 3 | Cohort 4 | |
|---|---|---|---|---|---|---|
| Failure | | 25 μg/25 mL | 6.25 μg/25 mL | 12.5 μg/25 mL | 12.5 μg/10 mL | Overall |
| Success* | n (%) | 5 (100.0%) | 3 (50.0%) | 5 (100.0%) | 4 (66.7%) | 17 |
| | | | | | | (77.2%) |
| Failure | n (%) | 0 (0.0%) | 3 (50.0%) | 0 (0.0%) | 2 (33.3%) | 5 |
| | | | | | | (22.8%) |

*Comparisons were to Day 0. Each success definition included no increase in any individual score.

The results for the cystitis events survey are shown below in Table 13. The percentage of cats presenting a decrease of ≥2 points or ≥3 points on Day 28 did not vary across cohort except for Cohort 2 (6.25 μg/25 mL). In Cohort 2, the number of cats presenting with a decrease in ≥2 points on Day 28 was 2 times larger than cats with a decrease of ≥3 points (66.7% vs. 33.3%, respectively).

TABLE 13
Cystitis Events Survey by cohort

| RTX Dose (Cohort) | | Day 28 | |
|---|---|---|---|
| 25 μg/25 mL (1) | Cystitis Improvement ≥2 Point | 4/5 | (80%) |
| | Cystitis Improvement ≥3 Point | 4/5 | (80%) |
| 6.25 μg/25 mL (2) | Cystitis Improvement ≥2 Point | 4/6 | (66.7%) |
| | Cystitis Improvement ≥3 Point | 2/6 | (33.3%) |
| 12.5 μg/25 mL (3) | Cystitis Improvement ≥2 Point | 5/5 | (100%) |
| | Cystitis Improvement ≥3 Point | 5/5 | (100%) |
| 12.5 μg/10 mL (4) | Cystitis Improvement ≥2 Point | 6/6 | (100%) |
| | Cystitis Improvement ≥3 Point | 6/6 | (100%) |

VSOM and Cystitis Events Survey were combined and are shown in Table 14. When the results were combined for VSOM improvement of ≥2 and cystitis improvement of ≥3, the effectiveness decreased from 100% to 80% (see Cohort 1). When the results were combined for VSOM improvement of ≥2 and cystitis improvement of ≥2, the effectiveness decreased from 50% to 33.3% (see Cohort 2). Overall, the decrease was approximately 20% in Cohorts 1-2.

TABLE 14
VSOM and Cystitis Events Survey combined effect

| RTX Dose (Cohort) | Success | Day 28 | |
|---|---|---|---|
| 25 μg/25 mL (1) | VSOM Improvement ≥2 Point | 5/5 | (100%) |
| | Cystitis ≥2 and VSOM ≥2 Point | 4/5 | (80%) |
| | Cystitis ≥3 and VSOM ≥2 Point | 4/5 | (80%) |
| 6.25 μg/25 mL (2) | VSOM Improvement ≥2 Point | 3/6 | (50%) |
| | Cystitis ≥2 and VSOM ≥2 Point | 3/6 | (50%) |
| | Cystitis ≥3 and VSOM ≥2 Point | 2/6 | (33.3%) |
| 12.5 μg/25 mL (3) | VSOM Improvement ≥2 Point | 6/6 | (100%) |
| | Cystitis ≥2 and VSOM ≥2 Point | 6/6 | (100%) |
| | Cystitis ≥3 and VSOM ≥2 Point | 6/6 | (100%) |
| 12.5 μg/10 mL (4) | VSOM Improvement ≥2 Point | 4/6 | (66.7%) |
| | Cystitis ≥2 and VSOM ≥2 Point | 4/6 | (66.7%) |
| | Cystitis ≥3 and VSOM ≥2 Point | 4/6 | (66.7%) |

As a way of monitoring the safety of the experiment for the enrolled cats, a physical examination was performed on the cats on Days 14 and 28. Measurements for body weight, body temperature, heart rate and respiratory rate were recorded. Body weight was collected for all cohorts, while the remaining variables were collected for Cohort 1, which received the highest total dose of RTX (25 μg/25 mL). No clinically significant changes were observed in any of the variables. Results were also collected for complete blood count (CBC), serum chemistry, urinalysis and culture. No significant changes were observed.

All doses were overall well tolerated at the time of administration and through the 28-day follow-up period. One serious adverse event (AE) considered to be related to the treatment at the time of the administration was reported in this study (the death of the Cohort 1 cat discussed above). No other adverse event was reported in cats of this cohort. Adverse events in other cohorts were considered unrelated to the treatment or not serious.

Descriptive statistics (Tables 10 and 11) show a high rate of success in all groups when VSOM on Day 28 was compared to Day 0. A minimum success rate of 50% was achieved in Cohort 2 (6.25 μg/25 mL), 100% success was seen in Cohorts 1 (25 μg/25 mL) and 3 (12.5 μg/25 mL), and an intermediate success of 66.7% was observed in Cohort 4 (12.5 μg/10 mL). Although the cats in Cohort 4 received the same total dose as Cohort 3 (12.5 μg total), and a lower dose than Cohort 1 (25 μg total), the concentration of the solution used in Cohort 4 was the highest of all groups (1.25 μg/mL vs. 1, 0.25 μg/mL and 0.5 μg/mL). It is possible that the lower efficacy observed in Cohort 4 compared to Cohorts 1 and 3 was related to the higher concentration used in Cohort 4.

### F. Administration of RTX Through Urethral Catheterization to the Bladder to Manage Lower Urinary Tract Discomfort in Dogs with Bladder Transitional Cell Carcinoma (TTC)

Canine TCC is a tumor of the uroepithelium that can invade into the deep layers of the bladder and is metastatic to regional lymph nodes and distant locations such as liver, lungs and bone in up to 50% of dogs. Urinary discomfort manifested by dysuria, pollakiuria, and hematuria is common with this histology. It is challenging to manage with conventional treatment options, which may be multimodal and include non-steroidal anti-inflammatory drugs (NSAIDs), conventional chemotherapy and radiation therapy. Sustained symptomatic benefit from these treatments is challenging. The primary objective of this study was to assess the status of lower urinary tract signs associated with RTX intravesical therapy in dogs with bladder TCC. The secondary objective of this study was to assess the anti-cancer effect of RTX intravesical therapy through serial abdominal ultrasonography and safety. The study was designed as an open label, multicenter, clinical field study with no randomization.

The study animals were client-owned dogs that presented with a diagnosis of TCC exclusively associated with the bladder (metastasis was acceptable) with accompanying lower urinary tract clinical signs associated with bladder cancer, provided the bladder lesion did not prevent the passage of urinary catheter. Study animals had no concurrent anti-cancer therapy including chemotherapy, molecular-targeted therapy, immunotherapy, or radiation therapy or had failed anti-cancer therapy and showed persistent lower urinary tract signs. NSAIDs and other pain medications were acceptable so long as dogs showed persisting lower urinary tract signs and the medications were used for at least 14 days prior to enrollment.

Pre-treatment with diphenhydramine HCl intramuscular (2 mg/kg) was administered 30-45 minutes prior to RTX administration. Enrolled dogs were anesthetized and were monitored by auscultation and chest movement observation. Heart rate and oxygenation were monitored using a pulse oximeter throughout the duration of anesthesia. Blood pressure was monitored. Body temperature was maintained using a heated water blanket or equivalent thermal barrier (e.g., Vetko). Lactated Ringers Solution (LRS), or other medically appropriate fluid therapy, were used to maintain adequate hydration (approximately 2 mL/kg/hr intravenous (IV)) and a fluid bolus of LRS (2-5 mL/kg IV) as needed.

RTX was administered as a one-time treatment into the bladder on Day 0 with the option of repeating RTX at a future study day. A Foley urinary catheter was placed using a sterile standard technique. An abdominal ultrasound was concurrently performed to ensure that the tip of the catheter was in the bladder lumen. Urine was removed using a catheter tipped syringe.

RTX was diluted in 0.9% NaCl and then infused into the bladder. The catheter was then flushed with 5-6 mL of 0.9% NaCl to ensure the entire IVP volume was administered. The Foley catheter was capped and remained in place for a period of 30 minutes post-infusion. The bladder was then emptied prior to removal of the Foley catheter. The timing of recovery was managed on an individual basis, but it was recommended to initiate recovery approximately 10 minutes after RTX infusion.

RTX total dose ranged from 25-100 μg (1.17-5.21 μg/kg). RTX was instilled at concentration of 1.0 μg/mL 2.0 μg/mL or 2.5 μg/mL.

Sixteen (16) client-owned dogs were enrolled in the study. Of these, 8 dogs completed the study. RTX administration by total dose, dose by bodyweight and concentration are summarized in Table 15.

TABLE 15
RTX administration by total dose, dose
by body weight, and concentration

| | | | | | RTX |
|---|---|---|---|---|---|
| Animal | Study | Body Weight | RTX Dose | RTX Dose | Concentration |
| ID | Day | (kg) | (μg) | (μg/kg) | (μg/mL) |
| 1 | 0 | 9.1 | 25 | 2.77 | 1.00 |
| 2 | 0 | 38.8 | 50 | 1.29 | 1.00 |
| 3 | 28 | 40.7 | 50 | 1.23 | 1.00 |
| 4 | 0 | 25.0 | 50 | 2.00 | 1.00 |
| 5 | 0 | 19.1 | 50 | 2.62 | 1.00 |
| 6 | 0 | 42.6 | 50 | 1.17 | 1.00 |
| 7 | 0 | 32.9 | 100 | 3.04 | 2.00 |
| 8 | 0 | 5.4 | 25 | 4.63 | 2.00 |
| 9 | 0 | 5.6 | 25 | 4.46 | 2.00 |
| 10 | 0 | 9.0 | 25 | 2.78 | 2.00 |
| 11 | 0 | 8.1 | 25 | 3.09 | 2.00 |
| 12 | 0 | 11.3 | 50 | 4.42 | 2.00 |
| 13 | 0 | 15.4 | 50 | 3.25 | 2.00 |
| 14 | 28 | 11.2 | 50 | 4.48 | 2.00 |
| 15 | 84 | 5.0 | 25 | 5.00 | 2.00 |
| 16 | 0 | 9.2 | 25 | 2.72 | 2.00 |
| 17 | 28 | 9.3 | 25 | 2.69 | 2.00 |
| 18 | 56 | 14.8 | 50 | 3.38 | 2.00 |
| 19 | 0 | 44 | 100 | 2.27 | 2.00 |
| 20 | 0 | 4.8 | 25 | 5.21 | 2.50 |
| 21 | 0 | 8.3 | 25 | 3.01 | 2.50 |
| 22 | 28 | 8.3 | 25 | 3.01 | 2.50 |

Primary effectiveness variables: Evaluations of lower urinary tract signs, as reported by the owner, were used as the primary effectiveness endpoint for the study. The character of urination, frequency of urination (day), frequency of urination (night) and blood in urine (Knapp, 2010) were monitored. Blood in urine was assessed by owner through daily collection of first morning sample and the owner completed the “Owner urine collection log.” Comparisons were made for each parameter between Day 0 and subsequent study visits on Days 7 (±2), 14 (±2), 28 (±2), 56 (±2) and 84 (±2). Treatment success was defined as at least one grade improvement for each parameter.

Primary Effectiveness Variable—Change in lower urinary tract signs: Of the 16 dogs enrolled, 14 dogs had at least one study visit after Day 0 to assess change in lower urinary tract grades. Six (6) dogs (37.5%) received a second RTX treatment: four (4) dogs received their second treatment at Day 28, 1 dog was re-treated at Day 56 and 1 dog was re-treated at Day 84.

At each study visit, the investigator graded lower urinary tract signs with input from the owner. Improvement in 1 or 2 grades are summarized by total RTX dose in Table 16 and then comparing those dogs receiving an RTX dose <3 μg/kg and 3 μg/kg in Table 17.

Two definitions of success were applied to the data: improvement in at least 1 score and improvement in at least 2 scores. Improvement in at least 1 score was reported for 10/14 dogs (71.4%) at Day 14, 8/11 dogs (72.7%) at Day 28, 7/10 dogs (70.0%) at Day 56 and 4/7 dogs (57.1%) at Day 84. Improvement in at least 2 scores was reported for 7/14 dogs (50.0%) at Day 14, 5/11 dogs (45.5%) at Day 28, 5/10 dogs (50.0%) at Day 56 and 3/7 dogs (42.9%) at Day 84. Of the 6 dogs that received a 2nd RTX administration, only 1 dog, retreated at Day 28, showed evidence of improved urine scores. This dog had improvement in 2 scores at Day 84 compared to Day 56.

TABLE 16
Change in lower urinary tract grades by RTX dose

| RTX | | Study Day: n/N (%) | | | | |
|---|---|---|---|---|---|---|
| Dose | Success | Day 7 | Day 14 | Day 28 | Day 56 | Day 84 |
| 25 μg | Improvement | 3/7 | 6/7 | 5/7 | 4/6 | 3/4 |
| | of at Least | (42.9%) | (85.7%) | (71.4%) | (66.7%) | (75.0%) |
| | 1 Score | | | | | |
| | Improvement | 1/7 | 3/7 | 2/7 | 2/6 | 2/4 |
| | of at Least | (14.3%) | (42.9%) | (28.6%) | (33.3%) | (50.0%) |
| | 2 Scores | | | | | |
| 50 μg | Improvement | 3/5 | 3/5 | 3/4 | 3/4 | 1/3 |
| | of at Least | (60.0%) | (60.0%) | (75.0%) | (75.0%) | (33.3%) |
| | 1 Score | | | | | |
| | Improvement | 2/5 | 2/5 | 3/4 | 3/4 | 1/3 |
| | of at Least | (40.0%) | (40.0%) | (75.0%) | (75.0%) | (33.3%) |
| | 2 Scores | | | | | |
| 100 μg | Improvement | 1/2 | 1/2 | | | |
| | of at Least | (50.0%) | (50.0%) | | | |
| | 1 Score | | | | | |
| | Improvement | 1/2 | 1/2 | | | |
| | of at Least | (50.0%) | (50.0%) | | | |
| | 2 Scores | | | | | |

TABLE 17
Change in lower urinary tract grades by RTX μg/kg

| RTX Dose | | | | | | |
|---|---|---|---|---|---|---|
| (ug/kg) | Success | Day 7 | Day 14 | Day 28 | Day 56 | Day 84 |
| <3 μg/kg | Improvement of at Least 1 Score | 4/7 | 5/7 | 4/5 | 4/5 | 2/2 |
| | | (57.1%) | (71.4%) | (80.0%) | (80.0%) | (100.0%) |
| | Improvement of at Least 2 Scores | 2/7 | 3/7 | 3/5 | 3/5 | 2/2 |
| | | (28.6%) | (42.9%) | (60.0%) | (60.0%) | (100.0%) |
| >= 3 μg/kg | Improvement of at Least 1 Score | 3/7 | 5/7 | 4/6 | 3/5 | 2/5 (40.0%) |
| | | (42.9%) | (71.4%) | (66.7%) | (60.0%) | |
| | Improvement of at Least 2 Scores | 2/7 | 3/7 | 2/6 | 2/5 | 1/5 (20.0%) |
| | | (28.6%) | (42.9%) | (33.3%) | (40.0%) | |

Secondary effectiveness variables: Anti-cancer activity was determined based on objective response (OR) using serial abdominal ultrasonography. The canine Response Evaluation Criteria for Solid Tumors (cRECIST) v 1.0 (Nguyen et al., 2013) was used to assess treatment response during the study. Longest diameter of the bladder lesion was recorded at the time of ultrasound. OR was defined as complete response (CR) or partial response (PR). In addition, biological response included CR, PR and stable disease (SD). For determination of SD to qualify as a biological response, SD was determined at Day 56 or Day 84. In the event that a bladder lesions was “diffuse” and not measurable, the Investigator subjectively determined treatment response in communication with the ultrasonographer.

Secondary Effectiveness Variables—Objective Response and Changes in QOL Tumor measurements (longest diameter) using ultrasound were reported at screening (baseline) and then at Day 28, Day 56 and Day 84. Objective response was defined as CR or PR and biological response as CR, PR, or SD. A summary of OR for the measurable target lesion in the bladder is provided in Table 18.

Of the 10 dogs that were evaluated with repeated abdominal ultrasound at Day 28, no OR were noted. However, one dog (11.1%) had a PR at Day 56 that continued until Day 84. SD for at least 28 days is considered clinically significant in dogs with bladder TCC. Eight (8) dogs (80.0%) had SD at Day 28, 7 dogs (77.8%) had SD at Day 56, and 3 dogs (42.9%) had SD at Day 84.

TABLE 18
Summary of objective response for all dogs

| | Study Day: n/N (%) | | |
|---|---|---|---|
| Response | Day 28 | Day 56 | Day 84 |
| CR/PR | 0/10 (0.0%) | 1/9 (11.1.0%) | 1/7 (14.3%) |
| SD | 8/10 (80.0%) | 7/9 (77.8%) | 3/7 (42.9%) |
| PD | 2/10 (20.0%) | 1/9 (11.1%) | 3/7 (42.9%) |

Adverse effects (AE): The most common AEs in the 16 dogs were emesis in 6 dogs (37.5%), diarrhea in 5 dogs (31.3%), anorexia in 4 dogs (25.0%), and lethargy in 4 dogs (25.0%). All of these events were considered mild to moderate (Grade 1 or 2) except one (1) anorexia event that was considered severe and resulted in the dog being euthanized.

Improvement in clinical signs were observed in this study of 16 dogs. For dogs that were evaluable at Days 14-84, more than 50% of these dogs had improvement in at least 1 urinary symptom grade and more than 40% had improvement in at least 2 grades at each visit. The improvement in symptoms at Day 84 is particularly significant given the potential for sustained symptom relief of RTX after only 1 or 2 treatments. Although 6 dogs received a second RTX administration, only 1 showed evidence of improvement in urinary tract signs after the second treatment.

RTX was very well tolerated in this study given small numbers of severe AE and only 1 dog with an SAE, considered most likely related to tumor progression. The 3 most commonly reported AE were gastrointestinal in nature (emesis, diarrhea, and anorexia) and all but 1 of these AE were mild or moderate and did not appear to be dose dependent.

What is claimed is:

1. A method of treating bladder pain or bladder cancer, comprising intravesically administering resiniferatoxin (RTX) to a subject in need of treatment of bladder pain or bladder cancer, wherein the RTX is administered at a dose of at least about 10 mcg or at least about 0.1 mcg/kg, or the RTX is administered at a concentration and volume such that the intravesical concentration of RTX in the bladder is at least about 0.1 mcg/ml.

2. A composition comprising resiniferatoxin (RTX) for use in a method of treating bladder pain or bladder cancer, the method comprising intravesically administering RTX to a subject in need of treatment of bladder pain or bladder cancer at a dose of at least about 10 mcg or at least about 0.1 mcg/kg, or the RTX is administered at a concentration and volume such that the intravesical concentration of RTX in the bladder is at least about 0.1 mcg/ml.

3. The method or composition for use according to claim 1 or 2, wherein the bladder pain comprises maladaptive bladder pain.

4. The method or composition for use of any one of the preceding claims, wherein the treatment reduces local and central effects of the maladaptive pain.

5. The method or composition for use according to any one of the preceding claims, wherein the subject has cystitis.

6. The method or composition for use according to any one of the preceding claims, wherein the subject has idiopathic cystitis.

7. The method or composition for use according to any one of the preceding claims, wherein the subject has bladder cancer.

8. The method or composition for use according to any one of the preceding claims, wherein the bladder pain comprises neuropathic bladder pain.

9. The method or composition for use according to any one of the preceding claims, wherein the bladder pain results from or is associated with stress-based activation of C fibers.

10. The method or composition for use according to any one of the preceding claims, wherein the subject previously underwent bladder surgery.

11. The method or composition for use according to any one of the preceding claims, wherein the bladder pain is subsequent to an injury, which is optionally an injury to the spine or lower back, such as a spinal disk injury.

12. The method or composition for use according to any one of the preceding claims, wherein the subject has experienced a bacterial bladder infection, or a plurality of bacterial bladder infections.

13. The method or composition for use according to any one of the preceding claims, wherein the subject has a hyperreactive bladder and/or a lowered threshold for bladder contraction and/or urge to urinate.

14. The method or composition for use according to any one of the preceding claims, wherein the method comprises administering RTX at a concentration of 0.1 mcg/ml-0.2 mcg/ml, 0.2 mcg/ml-0.3 mcg/ml, 0.3 mcg/ml-0.4 mcg/ml, 0.4 mcg/ml-0.5 mcg/ml, 0.5 mcg/ml-0.6 mcg/ml, 0.6 mcg/ml-0.7 mcg/ml, 0.7 mcg/ml-0.8 mcg/ml, 0.8 mcg/ml-0.9 mcg/ml, 0.9 mcg/ml-1.0 mcg/ml, 1.0 mcg/ml-1.1 mcg/ml, 1.1 mcg/ml-1.2 mcg/ml, 1.2 mcg/ml-1.3 mcg/ml, 1.3 mcg/ml-1.4 mcg/ml, 1.4 mcg/ml-1.5 mcg/ml, 1.5 mcg/ml-2 mcg/ml, 2 mcg/ml-3 mcg/ml, 3 mcg/ml-4 mcg/ml, 4 mcg/ml-5 mcg/ml, 5 mcg/ml-6 mcg/ml, 6 mcg/ml-7 mcg/ml, 7 mcg/ml-8 mcg/ml, 8 mcg/ml-9 mcg/ml, 9 mcg/ml-10 mcg/ml, 10 mcg/ml-11 mcg/ml, 11 mcg/ml-12 mcg/ml, 12 mcg/ml-13 mcg/ml, 13 mcg/ml-14 mcg/ml, or 14 mcg/ml-15 mcg/ml.

15. The method or composition for use according to any one of the preceding claims, wherein a dose of 10 mcg-20 mcg, 20 mcg-30 mcg, 30 mcg-40 mcg, 40 mcg-50 mcg, 50 mcg-60 mcg, 60 mcg-70 mcg, 70 mcg-80 mcg, 80 mcg-90 mcg, 90 mcg-100 mcg, 100 mcg-110 mcg, 110 mcg-120 mcg, 120 mcg-130 mcg, 130 mcg-140 mcg, 140 mcg-150 mcg, 150 mcg-160 mcg, 160 mcg-170 mcg, 170 mcg-180 mcg, 180 mcg-190 mcg, 190 mcg-200 mcg, 200 mcg-210 mcg, 210 mcg-220 mcg, 220 mcg-230 mcg, 230 mcg-240 mcg, 240 mcg-250 mcg, 250 mcg-260 mcg, 260 mcg-270 mcg, 270 mcg-280 mcg, 280 mcg-290 mcg, or 290 mcg-300 mcg of RTX is administered intravesically.

16. The method or composition for use according to any one of the preceding claims, wherein a dose of 0.1 mcg/kg-0.2 mcg/kg, 0.2 mcg/kg-0.3 mcg/kg, 0.3 mcg/kg-0.4 mcg/kg, 0.4 mcg/kg-0.5 mcg/kg, 0.5 mcg/kg-0.6 mcg/kg, 0.6 mcg/kg-0.7 mcg/kg, 0.7 mcg/kg-0.8 mcg/kg, 0.8 mcg/kg-0.9 mcg/kg, 0.9 mcg/kg-1 mcg/kg, 1 mcg/kg-1.2 mcg/kg, 1.2 mcg/kg-1.4 mcg/kg, 1.4 mcg/kg-1.6 mcg/kg, 1.6 mcg/kg-1.8 mcg/kg, 1.8 mcg/kg-2.0 mcg/kg, 2.0 mcg/kg-2.2 mcg/kg, 2.2 mcg/kg-2.4 mcg/kg, 2.4 mcg/kg-2.6 mcg/kg, 2.6 mcg/kg-2.8 mcg/kg, 2.8 mcg/kg-3.0 mcg/kg, 3.0 mcg/kg-3.2 mcg/kg, 3.2 mcg/kg-3.4 mcg/kg, 3.4 mcg/kg-3.6 mcg/kg, 3.6 mcg/kg-3.8 mcg/kg, 4.0 mcg/kg-4.2 mcg/kg, 4.2 mcg/kg-4.4 mcg/kg, 4.4 mcg/kg-4.6 mcg/kg, 4.6 mcg/kg-4.8 mcg/kg, 4.8 mcg/kg-5.0 mcg/kg, 5.0 mcg/kg-5.2 mcg/kg, 5.2 mcg/kg-5.4 mcg/kg, 5.4 mcg/kg-5.6 mcg/kg, 5.6 mcg/kg-5.8 mcg/kg, or 5.8 mcg/kg-6.0 mcg/kg of RTX is administered intravesically.

17. The method or composition for use according to any one of the preceding claims, wherein the RTX is delivered in a composition having a volume of 1 ml-10 ml, 10 ml-20 ml, 20 ml-30 ml, 30 ml-40 ml, 40 ml-50 ml, 50 ml-60 ml, 60 ml-70 ml, 70 ml-80 ml, 80 ml-90 ml, 90 ml-100 ml, 100 ml-110 ml, 110 ml-120 ml, 120 ml-130 ml, 130 ml-140 ml, 140 ml-150 ml, 160 ml-170 ml, 170 ml-180 ml, 180 ml-190 ml, 190 ml-200 ml, 200 ml-210 ml, 210 ml-220 ml, 220 ml-230 ml, 230 ml-240 ml, 240 ml-250 ml, 260 ml-270 ml, 270 ml-280 ml, 280 ml-290 ml, or 290 ml-300 ml.

18. The method or composition for use according to any one of the preceding claims, wherein the RTX is administered at a concentration and volume such that the intravesical concentration of RTX in the bladder is at least about 0.1 mcg/ml, such as 0.1 mcg/ml-0.2 mcg/ml, 0.2 mcg/ml-0.3 mcg/ml, 0.3 mcg/ml-0.4 mcg/ml, 0.4 mcg/ml-0.5 mcg/ml, 0.5 mcg/ml-0.6 mcg/ml, 0.6 mcg/ml-0.7 mcg/ml, 0.7 mcg/ml-0.8 mcg/ml, 0.8 mcg/ml-0.9 mcg/ml, or 0.9 mcg/ml-1 mcg/ml.

19. The method or composition for use according to any one of the preceding claims, wherein the RTX is administered at a concentration and volume such that the intravesical concentration of RTX in the bladder is at least about 1 mcg/ml, e.g., 1 mcg/ml-1.1 mcg/ml, 1.1 mcg/ml-1.2 mcg/ml, 1.2 mcg/ml-1.3 mcg/ml, 1.3 mcg/ml-1.4 mcg/ml, 1.4 mcg/ml-1.5 mcg/ml, 1.5 mcg/ml-1.6 mcg/ml, 1.6 mcg/ml-1.7 mcg/ml, 1.7 mcg/ml-1.8 mcg/ml, 1.8 mcg/ml-1.9 mcg/ml, or 1.9 mcg/ml-2 mcg/ml.

20. The method or composition for use of any one of the preceding claims, wherein the RTX is administered at a concentration of at least 0.25 mcg/ml.

21. The method or composition for use of any one of the preceding claims, wherein the RTX is administered at a concentration of at least 0.5 mcg/ml.

22. The method or composition for use of any one of the preceding claims, wherein the RTX is administered at a concentration of at least 1.0 mcg/ml.

23. The method or composition for use of any one of the preceding claims, wherein the RTX is administered at a concentration of at least 2.0 mcg/ml.

24. The method or composition for use of any one of the preceding claims, wherein the subject is a mammal.

25. The method or composition for use of claim 24, wherein the mammal is a cat or dog.

26. The method or composition for use of claim 24, wherein the mammal is a human.

27. The method or composition for use of claim 26, wherein the RTX is delivered in a composition having a volume of 50 ml-60 ml, 60 ml-70 ml, 70 ml-80 ml, 80 ml-90 ml, 90 ml-100 ml, 100 ml-110 ml, 110 ml-120 ml, 120 ml-130 ml, 130 ml-140 ml, 140 ml-150 ml, 160 ml-170 ml, 170 ml-180 ml, 180 ml-190 ml, 190 ml-200 ml, 200 ml-210 ml, 210 ml-220 ml, 220 ml-230 ml, 230 ml-240 ml, or 240 ml-250 ml.

28. The method or composition for use according to any one of the preceding claims, wherein the method comprises administering a pharmaceutical formulation comprising the RTX and a pharmaceutically acceptable carrier.

29. The method or composition for use of claim 28, wherein the pharmaceutically acceptable carrier comprises water.

30. The method or composition for use of claim 27 or 28, wherein the pharmaceutically acceptable carrier comprises polysorbate 80.

31. The method or composition for use of any one of claims 28-30, wherein the pharmaceutically acceptable carrier comprises a buffer, optionally wherein the buffer is phosphate buffer and/or the pH of the formulation is about 7.0-7.5 or about 7.2.
